SEQUENCE LISTING (4 sequences)

SEQ ID NO: 1 - protein, 142 aa, Homo sapiens
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 2 - protein, 173 aa, Homo sapiens
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 3 - protein, 143 aa, synthetic construct
GVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 4 - DNA, 12847 bp, Homo sapiens
  source 1..12847: mol_type = genomic DNA
acgtgtccctcgccgcgccccgtctacccgcccctgccctgaggaccctagtccaacatggcggcgcccagcggagggtggaacggcgtcggcgcgagcttgtgggccgcgctgctcctaggggccgtggcgctgaggccggcggaggcggtgtccgagcccacgacggtggcgtttgacgtgcggcccggcggcgtcgtgcattccttctcccataacgtgggcccgggggtacgtgccaccgccgtcccggatatttgagtgctggcaggcccggggagggggcgcggagcattgggggcggggcccgaggagggggcgcggaccgcggaggtgggtactgaagaaggggtgcggattgttagggtcggaggctgagtggggggcgcgggaccgtgaggccggggtcatcactggggcaggactgaactcatagggagcggggccttgtgggccccggcggcgttgagggggcgtgatcacctgtcacctgtcgcctgcctggagtgtccaggcacccctgccctccagacgcagaggtggggaggggtcagtgatgggagcggcaggaggctaaagggcatctgccctcctgggcctgaaggaaatcagtcgctgcagctccgaagagggaggaagtgctccttccttccattgcacaaggtcccccctcaccgagggtgtgaggccgggccctcagccctgtactcagcttgtcggcaaagtcctttcccctctcagcctccctttccccgcctgtaataacagtatctttactgttttttgactatttctttttagacttgcgtgcagtggcgggatctcggctcactgcatccttagccttctgggttcaagcaattcttctgcctcagcctccccagtacctgggattacagacgtgtgccaccacacccggataattgttgttgttgttgtttgagatggagcctcactctgtcgcccaggctggagtgcagtggcgcgatcttggctcactgcaacctctgcctcccaggttcatgccattctcctgccgcagcctcccaagtagctgggactactaatctttgtatttttagtacagatgggatttcaccatgttagccaggctggtcttgaactcctgaccgcaggtgacccgcccacctcggcctcccaaagtgatgggattacaggcgtgagccactgtgccaggccagtttgttgactacttccttatagttaggtgtgatgccaagtcctttatatgttaggcccaagttacagatgagaaaatgcttcacataaaaaagagaaacagagagaaactagaggctcagaaaagttttcacttgtccaggtcccatggcaaaacccatctctacaaaaatacgaaaaattagccaggtgtggtagcaggtgcctgtaatcccaagtactcaggaggctgaggcaggagaatcgtttgaacccgggaggcagaagttgcagtgagctgagatcatcccacttcagcctgggcgacagaacaagactctgcttcaaaaaaaaaaaaaaaaattagtcgggtgtggtggttgcgtacctattgtctcagttacttgagaggctgaggcaagaggattactcaaacctgggagttcgaggctgcagtgagctgtcattgtgcccctgcactccagcttgggtgacagcaagaccccgtctcttaaacaaaataaaaatttccttctaccaaaccttttttttccttccattctctaggacaaatatacgtgtatgttcacttacgcctctcaaggagggaccaatgaggtgagttgttcaaaattccctctagcttactgtgacagtatccattgttgtagggagatgttctgtagcagacagcagggttgggtctcacctatatcaccttacactcattgaatgactcttcctaaaccaataaaaagggagagtattggcattaaacagtgacgacagattctgctgttattattctacagagcaagcaaatgtctggacagaaggagggaggggagatgggagtctgccatccccatgagcatctgttgggatgttgtattttatgtgcagtgcaacaagttatcacagcttaggggcttaaaacagcacccatttattatctcatagctttgtaggtcagaagtctgggtcagctcagttgtgtgcttcataaaaccaaactcaaggtattttgggaggctgaggcgggcagatcacctgaggtcaaagttcgagaccagcctggccaacatggcaaaacctcatctctactaaaaatacaaaaattagctgggcatggtggcacatgcctgtagtcccagctactcaggaggctgaggcaggagaatcacttgaacccaggagacggaggttgcagtgagccaagattgtgccactgcactccagcctgggcaacagagcgagactctgtctcaaaaaataaataataaataaataaaggtatcacaggcaaggcatggtggctcatgccccatctctacagaaaataaaaaaattagcccagcgtggtggcgtgcacctgtggttccagctacttgggagactgaggtgggaggatcgcttgagcccaggaggtcaaggctgtagtgagccgtgattgtgctactgggcaacagagcaagaccctgtctcaaaaaaaaaaaaagtctctgggctgggagaagaatcctctccttcaagctcttagttggcagaattcagttacttgtggttcagggaccaaggtcccggtttccttgctggctgttgtctccagctggtggcagaagagccagtaaagatgatttgggccaggcacagtggctcatgcctataatcccagcactttgggaggccaaggctggcgaatcacctgaagtcaggagctccagaccagcctggccaacatggtgaaaccccgtctctactaaaactacaaaaattagccgggcgagccaggcgcactggctcatgcctgtaatcccagcactttgggaggccgaggcgggtggatcacaaggtcaggagatcgagaccatcctggctaacacggtgaaaccccgtctctactaaaaatacaaaaaattagccgggtgttttggtgggcacctgtagtcccagctactggggaggctgaggcaggagaatggcgtgaacccgggaggcggagcttgcagcgagccaagatcgcgccactgcactccatcctgggcgacagagcgagactctgtctcaaaaaaaaaaaaaaaaaaaaaaaaagggtgatttgaagcacatggacacatacgccactgctgcccttgagggaactgcagggcagggaatacacgcggcctctaggagctgagaacagcctctggctggcagcaaggaaatgaagacttcagtcccgcaaccaagaggaactgaaatctgccacaaccacaagagcttggaagaggaccttgagctaccagtaagagcctagcccggtcaacaccttgagatgggtttcgccatgttggccaggctgatcttgaactgctgacctcaaatgatcagcctgcctcggcctcccaaagtgctgggattacaggtgtgagccaccggtgcccggcctcctagagatttttctgtatcctatgtaaagcgcttcagttgcatggacgtgtggagttgacaatcccgtactaatggattgagggttacttccaacccagtccgtgaataaccttggccagggttcattgtacaagtgcagttacatctgtgggaaaaccacccagaagtgggattcatgggtccattttcttaaaagtaaaactgggctgggcgcggtggctcacgcctgtaatcccagcactttgagaggccaaggcaggtggatcgcctgaggttgggagtttgagaccagccttgctaagatggtgaaaccccatctctactgaaaatacaaaagttagcagggcgtggtggcacgcgcctgtaatcccagctactcaggaggctgaggcaggagaatcgcttgaacccacgaggcagaggttgcagtgagctgagatcgcaccactgcactccagcctgggtgacagagcgagactccatctcaaaaaaaaaaaaaaaagtaaaactgtatgttttgagatcatggtagatttacattcacttgtaagaaatcctacagagagatccaagtaccgtttaaccaggttccccggtggtaacatcgtgcaagaggccagtcattttagagatggggtctcgctatgttgcccaggctggtctcgaactcttgggttcaagtgatcatcccacgttggcctcccaaagtgctgggattacaggcatgagccaccatgcccagcctgcattttccattttgcttgtacctgttgcagctccaccgacaacctggaatagtcttggttttcacacccatatggtgtgttagcagacaggagggcttttgcccttctgacctgggagaaaaaggtatctcacgatagttcagcttgtgtttatttatttttcataaatgatatgggacatctttttttttttttttttttttttttttttttgagacagagtctcgctctgtcgcccaggctggagtgcagtggcgcgatctcagctcactgcaagctccgcctcccgggtacacgccattctcctgccttagcctcccgagtagctgggactacaggcgcccaccactgcgctcggctaattttttgtatttttagtagagatggggtttcatcatgttagccaggatggtcttgatctcctgacctcgtgatctgcctgcctcggcctcccaaagtgctgggattacaggcgtgagccaccgcgcccggcctggacatctatttatatgtttaagagttgttcttaggcctggcatggtggcttatatctgtaatctcagcactttgggaggccaaggtgggaggattgcttgaggccaggaggttgagaccagcctgggcaatatggcaagacagtgattctacaaaacacaaaaattagctgggtgtggtcccagctacttgggaggctgaggtgggaggatcgcttgagcccaggaggtagaggttgcagtgaggcgagatcgtgccactgcacgccagcccaggccacagagagagactccatctcaaaaacaaacaaacaaacaaacaaacagcgttgttcctatttccccatccgtgggagccactttggggtagttctcattagaggcaccacttttatgggaaggatgtggtgcaggcaggctccctgatcacagatgacagcaaaccccaggccaccaggagggcggggcaggggatttagtggccagcccctgaatcgggaggtacagaagaggtggcctgactgtagaatcagaggcacacctagaagtctccgagagcagctgtgtgtccggggctgacccgcgctgtcttctctccccagcaatggcagatgagtctggggaccagcgaagaccaccagcacttcacctgcaccatctggaggtgagactcggggtggggatggcatttccggctgccgttggcctgccctgtggcctcaggagctgctttggaaggcagggaacgagggctgcacccctggaggggaagacagaccagcagggagactcatgcacgtgcaggtggggggtggcagggtcgctggggtgggagtgctccccaaggaagtgcactgatgcagagactgagaaggtctgggggagacggggggagcctgtgcggggcaaggtcccaggaaaaggagaagcagctcagagggtgcggagcgtggaccagggcccaggaaggccccaaggggctccaccctcccctaagggtgagacgaactcaccaaagggctttttggcaatttttttttttttttttttgagatggagtctccatcttttgcccgggctgtagtgcagtggagcaatcttggctcactgcagcctccgcctcctgggttcaagcgattctcctccctcagcctcctgagtagctgggattacaggcgtgtgccaccatgcctgaccctttttggcaatgtttaaaaactgagataaaattccataaagcacagcattttaagctcacaattcagctgcatggagtactttctcagaggtgtgcagccagacctctgcttccagaacattttcatcccctgaaaataaactctgttcccatcagagtcattctccttcccagcacatggcagccacgcatccccctcctgtctctgtggatgggcgtgtcctggacattgcatagaaatggggtcaaaccttccctgtgtggcctttggtgtctggcgtctctcactgaacgtgacgtcctcaaggctcatccatgctgaggcccgtgtcagcctcatttcttttcctggctgagtcctattccagagggctccctgcaggaacatgtggagctcagatccccccggctgaaggaggcaggctgaggcagggagcccctccaggcaccaaccaggatggagtcgggggttgggggtggggcgcagattgaagcctgtgcagaatgaggatggggtgggtggagagggcacagattggaggctgtgtagaatgaggatggggtgggtggggagggtagattggaggctttgtagaatgaggatggggtgggtggggagagcacagattggagactgtgtagaatgaggacagggtgggtggggagggtatactggaggctgtagaatgaggatgaggtggggagggtagattggaggctgtagaatgaggatggggtgggtggggagggtagattggaggctgtgtagaatgagggtggggtgggtggggaggatagattggaggctgtgtagaatgaggatggggtgggtggagagtgcacaggttggaggctgtagaatgaggatggggtggggagggtagattggaggctgtgtagaatgaggatggggtgggtggagagggcacagattggaggctgtgtagaatgaggatggggtaggtggggagagtagattggatgctgtagaatgaggatggggtggatgggagcgtagattggaggctgtgtagaatgaggatgggatgggtggagagggcacagattggaggctgtagaatgaggatggggtgggtggggagggtagattggaggctgtgtagaatgaggacaggacgggtggggagggtagattggaggctgtgtagaatgaggacaggacgggtggggagggtagattggaggctgtagaatgaggatgaggtgggtggggaatgtagatcggaggctgtagaatgaggatggagtggggtggggagagtagattggaggctgtagaatgaggacggagtgggaggggaaggcacagactggaggctgtgtaaaatgaggacagggaggaggggagggcacagattggaggctgtggaatcagctagctggggtaggaggaggaggaaggtgctgggcagagtcctggtggaactggcggagctggtgcaggtctgggctggggacgagctaggcaggtggcaggtgtgggagtctgtggggtagtggctgtggaggtttggatgatgctatctcccggggaatgtgtcccaccagaaggggccccagctgtttcccctcaggcaggtctttaccagcatgttctctgaaggaccagcctgatggctcatagccttcttggggtgcagacatctgggaatcagacaaaagctgtgcactctcttccacctgcccgaacacacatgcatctgggtccacagcccctccccaagtctccccagacaccgcccgattctgcccctgccgccatcctctgtcaggctcatggtttatgggcttatcccggctccactcgccccactcattctcgtaaccccactgcctagatcccagctcgatctgggcaaggtcatgaagggaaggaagccgtcaggccccccggtggggagggggtcgggggcttcaggaatcttgtcccttcttccctgatccttggcccagaatgtctcagcccaaacagtgggtatttcttgagcctttattttgtgcagacactttggagggagaagtccaagctatagctccaccactgcggtcaggttaaagcacgttgacaaatgggaggcagagatcagggagtggcgacgtttaactgaaggacagggtgtgatttgctgcctcgtgtgggtgttgtcccgccgtgccctgcggggcgctttacaaacagcaggtgtttctggggagattctcacccactccatggaactggctgcatcacaggcaggccaaggccccaggaggcatcaggagggtcattgagaggagataggccccatcagcccagagagaccctgcagaggccgccctgtagtcttggggaagcccacggtcgccatcgtctgtctgcaggccactgccctgcatggacccctgagtgccagctccccagagggcatctgggcaggtgcaggtctggagcagttgggatggtggctgggctcaccttgagcctgaatcctcctgtccaacccccacaaaagctccccaggggcatgctcagccccggagacctgccaccccaggcctcctcatctcagacagtgactggaccctccctgaccagggctgttggcagtgctagggcttaaagaagaatctagggctccctggatacccccgccccacccatgcacatagaaccccagccagggcttccacagaaatggcccccctgctgcccctccatccccatcacactctcttcacaccagagatgccatcatcccctgctgtcaccttccgacaccttctgccactccagttccttagaagcctccgttagcagccttctagagacctgggcagatgatcctccccacttcttaagttaactcctgtacttcctagtcaatgccctctgagaagcctttactggcctctcattgcccagccccagcccccgtcgtagtacctggattgctgtcagcaaccaagaagccatgtgcagtgaatggacatggggtcctgtcccagagcagctcccagcccacaggggagagaggcgtgtctatgaatgagctggaagaacattctggcacaaaggggccaggcacagtggctcacacctctaatcacagccctctgggaggctgaggcgagtggatcacttgaggtcaggggttcaggccaggctggccaacatggtgaaaccatgtttttactaaaaataccaaaattagtcagatgtggtcgcgggtgcctgtactcccagctacaagggaggctaaggcaggagaatcacttaaacccaggaggcagaggctgcagtgagtgagattgtgccattgccatccagcctgggcaacagagtgagactcgtctccaaaaaaaaaaaaaaaaaaaaattctggcacaaaagaaagagggtctgctgttgagagggccccatccatccttcccagtcagcaggtgctcgtgctcgggaatggcgtccatgttgagaagcaggcacgagacctgctgaccccagcccgagtccctggcggggctccttccagggcacacagacccagacccagcaggcctggcctgactccctcgccccctcttcctctgcaggccccaggggaagtcctatctgtacttcacacagttcaaggcagaggtgcggggcgctgagattgagtacgccatggcctacgtgagtatcttggagtggggcaggctccgggtgtggcttctgtgcactggggcagctgtgagatgtcctgccacaagagggctccatggggacagggaggagaggggacagcgaatcctgcagaggtagggtgtgtaacttgcaggaacctggcacacagacgcccccaggccaggggcacgggggtttggtaacctgttgctgctggcttctcttgtatatgccagaaaagacttctccaggccaggcgcggcggttcacgccagtaatcccagcaatttgggaggctgaggcaggaggattgctggaggccaggaatttgattccagcctaggcaacatagcaagaccctatatctaccaaaagtaaaaaaaaaatagccaggcgtgttggcatgcacccatagtcccacctactcaggaggccgcagcaggaggatcacttgagtccaggactttgaagctgcagtgagctatgattgcactgctgcattccagcctggacagtagagtgaggtcctggctcaaaaacaaaaaagtctcctctccatctctagtaaaagtagaaagctttagtctgtgcttctctggaaagcaaactcaatcgttttcctaatgatgagcattgaactggccctggtaaagtttccactcattctttttcagtctaaagccgcatttgaaagggaaagtgatgtccctctgaaaactgaggaatttgaagtgaccaaaacagcaggtacggaaagatggggatggggtagaggtgacgccacccccatccaagggtgggcaatcggggcagtttggagaggggggtcagccataggggtcctggagactgtagaccaggcatccacaaactgattctataaaggccacctcgtcaatatcttcagctttgtggcactgtgggttctgtctagagctgtgccattgtggcacaaagctgctgcagacagtatgtccatgcgtgggtgtggctgtgtgccactagaactttatcttagctgggcgcggtggctcatgcctgttaccgcagctgctcaggaggctaaggtgggaggatcattggagcccaggaggtcaagaccagcctgggcaacatagcgagaccctgtctctacaaaatttttttaaaaatcaggctgggtgcggtggctcacgcctgtaatcccagcactttgggaggacgaggcgggcggaacacttgaggtcaggagttccagaccagctggccaacataatgaaaccccatctctactaaaaatgcaaaaaattagccaggcgtggtggtgcgcacctgtaatcccagctattcaggagaattgctggaacctgggaggtggaggttgcagtgagctgagatcatgccactgcactctagcctggaagacagcaagattctgtctcaaaaagaaaaaaaattagccgggcatggtggtgtgtacctgtagtctcagctacttaggaggctgaggtgggaggattatttgagcccaggaggtcgaggctgcattgagctatgatcgcaccactgcactccagcctgggcatcagagtaagactctgtctcaaaaacaataaacagggctgggcatggtggctcacacctgtaatcccagcactttgggaggccaggagttccagaccagcctagccaacatggtgaaaccccttctctactaaaaataccaaaattagccaaggatggtggcacatgcttgtaatcccagctactcaggaggcagagacaggagaatctcttgaacttaggagcaggaggttgcagtgagtcgagactgcaccactgcactccagcctaggcaacagaggaagactctcatctcaaaaaaaataataaattttaaaaaataaataaatgaaataaaacaaagtagaaaccatcagggctttgaggttgggactgtggcatgccgaggggaggctgcaggggtcactgggaaacatcagggtccttcttcggttgcctgagcagcaccggtgtcctcaagccacattcagctgcatggcgccaatgctaagcagaaagaggaatcggaggcgccgagctggggcaagggtgggagccaagggagtggacacagcaggtagggccgggagacttgggtgaagcagtggaagccagagttctgaccctgctggagaagtgtcatgagggcctcagacaggttcacccactgagcaggaggtcttgcagcctgacccaccctgcatcctgttcaggccgggatcatctccttcctgggcttcttctggcccctttctgcatctcttcttgacaacatggcacaaatgaccagggttcttcccacctcaggtctttctgtggcttcctgctgcctgccatctatgttagccccaccgaccttaccctccacatcactgttttcctgtctgtcacccgtaagactttcagtgcctggggccttttcacatgggagtctgctttggagactgctccccttgcctgcttagcccccaccatgggccccaccccaaatcctccggacttctgagaattttcaatgttgagggcccaaccatgtgtttctttcttgcagtggctcacaggcccggggcattcaaagctgagctgtccaagctggtgattgtggccaaggcatcgcgcactgagctgtgaccagcagccctgttgcgggtggcaccttctcatctccggtgaagctgaaggggcctgtgtccctgaaagggccagcacatcactggttttctaggagggactcttaagttttctacctgggctgacgttgccttgtccggaggggcttgcagggtggctgaagccctggggcagagaacagagggtccagggccctcctggctcccaacagcttctcagttcccacttcctgctgagctcttctggactcaggatcgcagatccggggcacaaagagggtggggaacatgggggctatgctggggaaagcagccatgctccccccgacctccagccgagcatccttcatgagcctgcagaactgctttcctatgtttacccaggggacctcctttcagatgaactgggaagagatgaaatgttttttcatatttaaataaataagaacattaaaaagcaa

(19) United States
(12) Patent Application Publication
(10) Pub. No.: US 2026/0337243 A1
(43) Pub. Date: Oct. 29, 2026

(54) MYELOID-DERIVED GROWTH FACTOR FOR USE IN TREATING CARDIOGENIC SHOCK

(71) Applicants: BOEHRINGER INGELHEIM INTERNATIONAL GMBH, Ingelheim am Rhein (DE); MEDIZINISCHE HOCHSCHULE HANNOVER, Hannover (DE)

(72) Inventors: Kai Christoph WOLLERT, Hannover (DE); Yong WANG, Hannover (DE)

(21) Appl. No.: 19/108,911

(22) PCT Filed: Sep. 8, 2023
(86) PCT No.: PCT/EP2023/074803

§ 371 Date: Mar. 5, 2025

Related U.S. Application Data
(60) Provisional application No. 63/374,942, filed on Sep. 8, 2022.

(51) Int. Cl. C07K 14/475; A01K 67/0276; A61K 38/00; A61P 9/04; A61P 9/10

(52) U.S. Cl. CPC C07K 14/475; A01K 67/0276; A61P 9/04; A61P 9/10; A01K 2207/30; A01K 2207/35; A01K 2217/075; A01K 2227/105; A01K 2267/0375; A61K 38/00

(57) ABSTRACT

The present invention relates to the protein myeloid-derived growth factor (MYDGF) or nucleic acids encoding said protein for use in treating and/or preventing cardiogenic shock. The present invention also relates to vectors comprising the nucleic acid, host cells expressing the nucleic acid, pharmaceutical compositions comprising said protein, nucleic acid, vector or host cell for use in treating and/or preventing cardiogenic shock, and to methods for treating and/or preventing cardiogenic shock. The present invention further relates to a method of preparing an animal model of cardiogenic shock, and to animals obtained by said method.

### SEQUENCE LISTING

The instant application contains a sequence listing which has been submitted electronically in xml format and is hereby incorporated by reference in its entirety.

### TECHNICAL FIELD OF THE INVENTION

The present invention relates to the protein myeloid-derived growth factor (MYDGF) for use in treating and/or preventing cardiogenic shock. The present invention also relates to a novel animal model of cardiogenic shock.

### BACKGROUND OF THE INVENTION

Myeloid-derived growth factor (MYDGF), also known as Factor 1, is a protein encoded in open reading frame 10 on human chromosome 19 (C19Orf10). The Protein was described in 2007 in a proteome-analysis of the so called fibroblast-like synoviocytes (FLS-cells) as a new secreted Factor in the synovium. A correlation between the secretion of the protein and inflammatory diseases of the joint has been supposed without any experimental or statistical evidence (Weiler et al., Arthritis Research and Therapy 2007, The identification and characterization of a novel protein, c19orf10, in the synovium). A corresponding patent application claims the protein as therapeutic agent for the treatment of joint and for the diagnosis of a tissue undergoing altered growth as well as monitoring changes in a tissue (US 2008/0004232 A1, Characterization of c19orf10, a novel synovial protein). Another scientific publication describes an enhanced expression of the protein in hepatocellular carcinoma cells (Sunagozaka et al., International Journal of Cancer, 2010, Identification of a secretory protein c19orf10 activated in hepatocellular carcinoma). Recombinant produced protein showed a proliferation enhancing effect on cultured hepatocellular carcinoma cells. It is noted that C19Orf10 has also been referred to as IL-25, IL-27 and IL-27W as it was originally considered an interleukin. However, the terms “IL-25” and “IL-27” have been used inconsistently in the art and have been used to designate a variety of different proteins. For example, US 2004/0185049 refers to a protein as IL-27 and discloses its use in modulating the immune response. This protein is structurally distinct from Factor 1 (compare Factor 1 amino acid sequence according to SEQ ID NO: 1 to the amino acid sequence of “IL-27” according to UniProt: Q8NEV9). Similarly, EP 2 130 547 A1 refers to a protein as IL-25 and discloses its use in treating inflammation. This protein has also been referred to in the art as IL-17E and is structurally distinct from Factor 1 (compare the amino acid sequence of Factor 1 according to SEQ ID NO: 1 to the amino acid sequence of “IL-25” according to UniProt: Q9H293).

WO 2014/111458 discloses Factor 1 for use in enhancing proliferation and inhibiting apoptosis of non-transformed tissue or non-transformed cells, in particular for use in treating acute myocardial infarction. Further disclosed are inhibitors of Factor 1 for medical use, in particular for use in treating or preventing a disease in which angiogenesis contributes to disease development or progression.

Korf-Klingebiel et al. (Nature Medicine, 2015, Vol. 21 (2): 140-149) report C19Orf10 to be secreted by bone marrow cells after myocardial infarction, which protein promotes cardiac myocyte survival and angiogenesis. The authors show that bone marrow-derived monocytes and macrophages produce this protein endogenously to protect and repair the heart after myocardial infarction, and propose the name myeloid-derived growth factor (MYDGF). In particular, treatment with recombinant mouse Mydgf is reported to reduce scar size and contractile dysfunction after myocardial infarction.

WO 2021/148411 discloses MYDGF for use in treating or preventing fibrosis, hypertrophy or heart failure. Heart failure is a clinical syndrome with a poor prognosis that may develop in response to persistent hemodynamic overload, myocardial injury, or genetic mutations.

Cardiogenic shock (CS) is a life-threatening, acute low cardiac output state resulting from severe systolic and/or diastolic myocardial dysfunction and leading to arterial hypotension, pulmonary congestion, critical end-organ hypoperfusion, and impaired tissue oxygenation. CS is associated with insufficient blood flow to the extremities and vital organs, including the heart itself, the liver, kidneys, and the brain. Critical end-organ hypoperfusion and impaired tissue oxygenation lead to an increase in blood lactate concentration, which is used for diagnosis and monitoring of patients with CS. Clinical criteria for defining CS are summarized e.g. in the review article of Vahdatpour et al. (Journal of the American Heart Association, Vol 8 (8), 2019, e011991). Hypoperfusion and impaired oxygenation of the heart itself lead to a progressive worsening of cardiac performance in CS thereby triggering a downward spiral of increasing hemodynamic instability associated with very high mortality.

Although CS may develop as a complication of myocardial infarction, the pathophysiologies of an uncomplicated myocardial infarction (without cardiogenic shock) and a (typically large) myocardial infarction with cardiogenic shock are largely different. Patients with an uncomplicated myocardial infarction do not present with critical end-organ hypoperfusion and impaired tissue oxygenation, they do not have an acute and progressive worsening of cardiac function (downward spiral), and they do not have the very high acute mortality observed in patients with CS.

Likewise, patients with heart failure, fibrosis and hypertrophy of the heart do not acutely present with critical end-organ hypoperfusion and impaired tissue oxygenation, they do not have an acute progressive worsening of cardiac function (downward spiral), and they do not have the very high acute mortality observed in patients with CS.

Exemplifying the distinct pathophysiologies of these conditions, medical therapies known to improve outcome in patients with uncomplicated myocardial infarction and in patients with heart failure, fibrosis and hypertrophy of the heart (e.g. angiotensin-converting enzyme inhibitors and beta-blockers) are not useful and even contraindicated in CS. In fact, so far, no medical therapies have been identified that can improve the survival of patients in CS.

Thus, there is an urgent need for means and methods for treating and/or preventing cardiogenic shock. There is also a need for suitable animal models of cardiogenic shock to further elucidate the underlying pathophysiology and to define novel therapies for this condition.

### SUMMARY OF THE INVENTION

The invention provides in a first aspect myeloid-derived growth factor (MYDGF) or a fragment or a variant thereof exhibiting the biological function of MYDGF, for use in treating and/or preventing cardiogenic shock.

According to a preferred embodiment, the MYDGF protein comprises SEQ ID NO: 1. According to one embodiment, the MYDGF protein comprises a fragment or variant of SEQ ID NO: 1, which exhibits the biological function of MYDGF, and which comprises an amino acid sequence having at least 85% amino acid sequence identity to SEQ ID NO: 1.

According to a particular preferred embodiment, the MYDGF protein consists of SEQ ID NO: 1 or SEQ ID NO: 3.

According to a further aspect, the present invention provides a nucleic acid encoding MYDGF or the fragment or variant thereof exhibiting the biological function of MYDGF, for use in treating and/or preventing cardiogenic shock.

According to one embodiment, the nucleic acid encodes an amino acid sequence having at least 85% sequence identity to SEQ ID NO: 1.

According to a further aspect, the present invention provides a vector comprising the nucleic acid of the present invention for use in treating and/or preventing cardiogenic shock.

According to a further aspect, the present invention provides a host cell comprising the nucleic acid of the present invention or the vector of the present invention for use in treating and/or preventing cardiogenic shock. Preferably, the host cell expresses the nucleic acid.

According to yet another aspect, the present invention provides a pharmaceutical composition comprising the MYDGF protein, the nucleic acid, the vector or the host cell of the present invention and optionally a suitable pharmaceutical excipient and/or carrier, for use in treating and/or preventing cardiogenic shock.

According to a preferred embodiment, the pharmaceutical composition for use is administered through the oral, intravenous, subcutaneous, intramucosal, intraarterial, intramuscular or intracoronary route. The administration is preferably through one or more bolus injection(s) and/or infusion(s).

According to a further aspect, the present invention provides a method of treating and/or preventing cardiogenic shock comprising administering to a patient in need thereof a therapeutically effective amount of myeloid-derived growth factor (MYDGF) protein.

According to one embodiment, the MYDGF protein comprises a fragment or variant of SEQ ID NO: 1, which exhibits the biological function of MYDGF, and which comprises an amino acid sequence having at least 85% amino acid sequence identity to SEQ ID NO: 1.

According to yet another embodiment, the MYDGF or fragment or variant thereof is administered through one or more bolus injection(s) and/or infusion(s), preferably in a pharmaceutically accepted carrier and/or excipient.

According to a further aspect, the present invention provides a method of treating and/or preventing cardiogenic shock comprising administering to a patient in need thereof a therapeutically effective amount of a pharmaceutical composition comprising myeloid-derived growth factor (MYDGF) protein or a fragment or variant thereof. According to one embodiment, the MYDGF protein comprises a fragment or variant of SEQ ID NO: 1, which exhibits the biological function of MYDGF, and which comprises an amino acid sequence having at least 85% amino acid sequence identity to SEQ ID NO: 1.

According to one preferred embodiment, the pharmaceutical composition comprises a suitable pharmaceutical excipient.

According to another embodiment, the pharmaceutical composition is administered through one or more bolus injection(s) and/or infusion(s).

According to a further aspect, the present invention provides a method for producing a non-human mammalian model of cardiogenic shock, the method comprising (i) transiently ligating a coronary artery of the mammal, (ii) establishing reperfusion, and (iii) ventilating the mammal with a fraction of inhaled oxygen of about 0.18 or less.

According to a preferred embodiment, the non-human mammal is a rodent, more preferably a mouse.

According to a preferred embodiment, the coronary artery is ligated for about 30 minutes to about 90 minutes before reperfusion is established.

According to a preferred embodiment, the mammal is ventilated with a fraction of inhaled oxygen of about 0.16.

### BRIEF DESCRIPTION OF THE DRAWINGS

FIG. 1: A: Schematic illustration of the process scheme for preparing the cardiogenic shock animal model. Myocardial infarction (MI) was induced in 8- to 10-week-old male C57BL/6N mice by proximal left anterior descending coronary artery ligation for 60 min. Animals were subcutaneously pretreated with 2 mg/kg butorphanol and with 0.02 mg/kg atropine to reduce bronchial secretions. Anaesthesia was induced with 3-4% isoflurane. After intubation, mice were mechanically ventilated and anaesthesia was maintained with 1-2% isoflurane. After reperfusion, a micromanometer-tipped conductance catheter was inserted via the right carotid artery to continuously record left ventricular (LV) pressure-volume (PV) loops. Subsequently, mice were ventilated for 120 min with a fraction of inspired oxygen (FiO₂) concentration of 0.33 or 0.16. As shown in pilot experiments, an FiO₂ of 0.33 was associated with normoxaemia [nx; arterial oxygen partial pressure (PaO₂), 144±16 mmHg; arterial oxygen saturation (SaO₂), 99±1%], whereas an FiO₂ of 0.16 resulted in mild hypoxaemia (hx; PaO₂, 75±16 mmHg; SaO₂, 89±3%) (4-6 mice per group). Subsequently, infarcted and sham-operated mice were randomly ventilated with either FiO₂ and followed for 120 min, as described in Wang et al. Cardiovasc Res. 2021; 117:2414-2415. B: left ventricular pressure volume loops sampled at 120 minutes of sham operated mice (sham) (dotted lines) and mice with induced myocardial infarction (MI) (uninterrupted lines) ventilated under normoxic (nx) and hypoxic (hx) conditions.

FIG. 2: Left ventricular end-systolic pressure (LVESP) in mmHg (FIG. 2A), cardiac output in mL/min (FIG. 2B), and arterial lactate concentration in mmol/L (FIG. 2C) of sham operated mice (sham) (open circles) and mice with induced myocardial infarction (MI) (filled circles) at 120 min, all ventilated under normoxic (nx) or hypoxic (hx) conditions, respectively. MI mice ventilated with an FiO₂ of 0.16 (MI-hx) progressively developed CS as defined by the clinical hallmarks of CS, i.e. hypotension (reduced LVESP), reduced cardiac output, and increased lactate concentration. MI mice ventilated with an FiO₂ of 0.33 (MI-nx) remained hemodynamically stable.

FIG. 3: Starting 60 min after the initiation of hypoxic ventilation, MI-hx mice were treated with an intravenous dobutamine infusion (5 to 7.5 ng/g/min via left jugular vein, titrated to a LVESP of 70 mmHg) (MI-hx, dobutamine). Saline-infused MI-hx mice served as controls (MI-hx, saline). Dobutamine improved LVESP (FIG. 3A) and cardiac output (FIG. 3B) in MI-hx mice. Similar hemodynamic improvements are typically observed in patients with CS treated with dobutamine.

FIG. 4: Phosphoproteome analysis using high-resolution mass spectrometry with unsupervised principal component analysis (6 mice per group) in the non-infarcted left ventricular myocardium. Four experimental groups were associated with distinct phosphoproteome signatures: Sham-nx=sham operated mice normoxaemia; Sham-hx=sham operated mice hypoxaemia; MI-nx=infarcted mice normoxaemia; MI-hx=infarcted mice hypoxaemia.

FIG. 5: Volcano plot illustrating differentially regulated phosphosites in the non-infarcted left ventricular myocardium of mice with myocardial infarction-induced CS (MI-hx) versus mice with myocardial infarction only (MI-nx). The top 10 down- or upregulated [P<0.05] phosphosites are shown in black. MI-nx=infarcted mice normoxaemia; MI-hx=infarcted mice hypoxaemia. Abbreviations denote proteins, amino acid positions indicated in brackets show the respective phosphorylated residue in each protein. R1PR1=Rho family-interacting cell polarization regulator 1. DDA1=DET1- and DDB1-associated protein 1. OSB11=oxysterol-binding protein-related protein 11. ODPA=pyruvate dehydrogenase E1 component subunit alpha. SCRIB=Protein scribble homolog. SRF=serum response factor. KCNH2=potassium voltage-gated channel subfamily H member 2. CSRP1=cysteine and glycine-rich protein 1. MYH6=myosin-6. DP13A=DCC-interacting protein 13-alpha. PDLI5=PDZ and LIM domain protein 5. TITIN=titin. HSPB1=heat shock protein beta-1. DESMIN=desmin. LMNA=prelamin-A/C. XIRP1=Xin actin-binding repeat-containing protein 1. TNNI3=troponin I. TIF1B=transcription intermediary factor 1-beta.

FIG. 6: Kaplan-Meier survival curve showing mortality over 120 minutes in cardiogenic shock (MI-hx) mice treated with MYDGF or with saline.

FIG. 7: Bar graphs showing results from PV loop recordings at the end of the 120 minutes observation period. Heart rate (FIG. 7A), left ventricular end-systolic volume (LVESV) (FIG. 7B), left ventricular end-diastolic volume (LVEDV) (FIG. 7C), cardiac output (FIG. 7D), stroke volume (FIG. 7E) and stroke work (FIG. 7F) of cardiogenic shock (MI-hx) mice treated with MYDGF or with saline. * P<0.05, ** P<0.01, *** P<0.001.

FIG. 8: Bar graphs showing results from PV loop recordings at the end of the 120 minutes observation period. Left ventricular end-systolic pressure (LVESP) (FIG. 8A), left ventricular end-diastolic pressure (LVEDP) (FIG. 8B), left ventricular ejection fraction (LVEF) (FIG. 8C), maximal rate of pressure change in the left ventricle (dP/dtₘₐₓ) (FIG. 8D), minimal rate of pressure change in the left ventricle pressure (dP/dtₘᵢₙ) (FIG. 8E), and left ventricular diastolic time constant (Tau) (FIG. 8F) of cardiogenic shock (MI-hx) mice treated with MYDGF or with saline. ** P<0.01, *** P<0.001.

FIG. 9: Bar graphs showing results from blood gas analysis at the end of the 120 minutes observation period. pH (FIG. 9A), arterial blood oxygen partial pressure (PaO₂ in mmHg) (FIG. 9B), arterial oxygen saturation (SaO₂ in %) (FIG. 9C), and lactate concentration (in mmol/L) (FIG. 9D) of cardiogenic shock (MI-hx) mice treated with MYDGF or with saline. * P<0.05.

FIG. 10: Results from continuous PV loop recordings during the 120 minutes observation period in the CS model. Cardiac output (in mL/min) (FIG. 10A) and left ventricular end-systolic pressure (LVESP) (in mmHg) (FIG. 10B) of saline-treated MI-hx mice (CS saline) versus MYDGF-treated MI-hx mice (CS MYDGF).

FIG. 11: Area at risk and infarct size of the left ventricle determined at the end of the 120 min observation period in the CS model. Saline-treated MI-hx mice (Saline) versus MYDGF-treated MI-hx mice (MYDGF). Area at risk/total LV area (in %) (FIG. 11A) and infarct size/area at risk (in %) (FIG. 11B).

FIG. 12: Plasma concentration of high-sensitivity cardiac troponin T (cTnT) and the liver damage marker alanine aminotransaminase (ALT) at the end of the 120 minutes observation period in the CS model. Left ventricular soluble nucleosome concentration at the end of the 120 minutes observation period in the CS model. cTnT (in ng/mL) (FIG. 12A) and alanine aminotransaminase (Log₂ALT in U/L) (FIG. 12B) in saline-treated MI-hx mice (Saline) versus MYDGF-treated MI-hx mice (MYDGF). Soluble nucleosome concentrations measured in the infarcted (I) versus non-infarcted (NI) regions of the left ventricle were determined by ELISA. The I/NI ratio serves as an indicator for cell death (FIG. 12C).

FIG. 13: Schematic illustration of the treatment regimen used on the cardiogenic shock animal model in the previous examples (FIGS. 6, 7, 8, 9, 10, 11, and 12). Abbreviations shown in figure are as defined for FIG. 1.

FIG. 14: Kaplan-Meier survival curve showing mortality over 120 minutes of cardiogenic shock of wild-type mice (WT) and Mydgf gene-deficient (knock-out) mice (MYDGF KO).

FIG. 15: Aggravation of left ventricular tissue damage caused by cardiogenic shock. FIG. 15A shows the experimental set-up. FIG. 15B shows the results of the experiment, indicating that cardiogenic shock further aggravates left ventricular tissue damage sustained during acute myocardial infarction.

FIG. 16: Delayed treatment of cardiogenic shock. FIG. 16A shows the experimental set-up. FIG. 16B shows the results of the experiment, indicating that cardiogenic shock can be treated by administering MYDGF at a time point after reperfusion has been established.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Definitions

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, cell biology, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989). Furthermore, conventional methods of clinical cardiology are employed which are also explained in the literature in the field (cf., e.g., Braunwald's Heart Disease. A Textbook of Cardiovascular Medicine, 9^{th} Edition, P. Libby et al. eds., Saunders Elsevier Philadelphia, 2011).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

As used herein, the term “and/or” means that it refers to either one or both/all of the options cited in the context of this term.

Nucleic acid molecules also termed nucleic acids are understood as polymeric macromolecules made from nucleotide monomers. Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA) and deoxyribonucleic acid (DNA). The terms “polynucleotide” and “nucleic acid” are used interchangeably herein.

The term “open reading frame” (ORF) refers to a sequence of nucleotides, that can be translated into amino acids. Typically, such an ORF contains a start codon, a subsequent region usually having a length which is a multiple of 3 nucleotides, but does not contain a stop codon (TAG, TAA, TGA, UAG, UAA, or UGA) in the given reading frame. Typically, ORFs occur naturally or are constructed artificially, i.e. by gene-technological means. An ORF codes for a protein where the amino acids into which it can be translated form a peptide-linked chain.

The terms “protein” and “polypeptide” are used interchangeably herein and refer to any peptide-bond-linked chain of amino acids, regardless of length or post-translational modification. Proteins usable in the present invention (including protein derivatives, protein variants, protein fragments, protein segments, protein epitopes and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility. Chemical modifications applicable to the variants usable in the present invention include without limitation: PEGylation, glycosylation of non-glycosylated parent polypeptides, covalent coupling to therapeutic small molecules, like glucagon-like peptide 1 agonists, including exenatide, albiglutide, taspoglutide, DPP4 inhibitors, incretin and liraglutide, or the modification of the glycosylation pattern present in the parent polypeptide. Such chemical modifications applicable to the variants usable in the present invention may occur co-or post-translational.

The term “amino acid” encompasses naturally occurring amino acids as well as amino acid derivatives. A hydrophobic non-aromatic amino acid in the context of the present invention, is preferably any amino acid which has a Kyte-Doolittle hydropathy index of higher than 0.5, more preferably of higher than 1.0, even more preferably of higher than 1.5 and is not aromatic. Preferably, a hydrophobic non-aromatic amino acid in the context of the present invention, is selected from the group consisting of the amino acids alanine (Kyte Doolittle hydropathy index 1.8), methionine (Kyte Doolittle hydropathy index 1.9), isoleucine (Kyte Doolittle hydropathy index 4.5), leucine Kyte Doolittle hydropathy index 3.8), and valine (Kyte Doolittle hydropathy index 4.2), or derivatives thereof having a Kyte Doolittle hydropathy index as defined above.

The term “variant” is used herein to refer to a polypeptide which differs in comparison to the polypeptide or fragment thereof from which it is derived by one or more changes in the amino acid sequence. The polypeptide from which a protein variant is derived is also known as the parent polypeptide. Likewise, the fragment from which a protein fragment variant is derived from is known as the parent fragment. Typically, a variant is constructed artificially, preferably by gene-technological means. Typically, the parent polypeptide is a wild-type protein or wild-type protein domain. Further, the variants usable in the present invention may also be derived from homologs, orthologs, or paralogs of the parent polypeptide or from artificially constructed variant, provided that the variant exhibits at least one biological activity of the parent polypeptide. The changes in the amino acid sequence may be amino acid exchanges, insertions, deletions, N-terminal truncations, or C-terminal truncations, or any combination of these changes, which may occur at one or several sites. In preferred embodiments, a variant usable in the present invention exhibits a total number of up to 23 (up to 1, 2, 3, 4, 5, 6, 7, 8, 9, 10, 11, 12, 13, 14, 15, 16, 17, 18, 19, 20, 21, 22, or 23) changes in the amino acid sequence (i.e. exchanges, insertions, deletions, N-terminal truncations, and/or C-terminal truncations). A particularly preferred variant of SEQ ID NO: 1 is shown in SEQ ID NO: 3, which exhibits one additional amino acid (glycine) at its N-terminus (+G variant). The amino acid exchanges may be conservative, and/or semi-conservative, and/or non-conservative. In preferred embodiments, a variant usable in the present invention differs from the protein or domain from which it is derived by up to 1, 2, 3, 4, 5, 6, 7, 8, 9, 10, 11, 12, 13, 14, 15, 16, 17, 18, 19, 20, 21, 22, or 23 amino acid exchanges, preferably conservative amino acid changes.

Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are:

| Amino | Conservative | Semi- |
|---|---|---|
| acid | substitution | conservative |
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure.

Alternatively or additionally, a “variant” as used herein, can be characterized by a certain degree of sequence identity to the parent polypeptide or parent polynucleotide from which it is derived. More precisely, a protein variant in the context of the present invention exhibits at least 85% sequence identity to its parent polypeptide. The term “at least 85% sequence identity” is used throughout the specification with regards to polypeptide and polynucleotide sequence comparisons. This expression preferably refers to a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

Fragments of proteins comprise deletions of amino acids, which may be N-terminal truncations, C-terminal truncations or internal deletions or any combination of these. Such proteins comprising N-terminal truncations, C-terminal truncations and/or internal deletions are referred to as “fragments” in the context of the present application. A fragment may be naturally occurring (e.g. splice variants) or it may be constructed artificially, preferably by gene-technological means. Preferably, a fragment (or deletion variant) has a deletion of up to 1, 2, 3, 4, 5, 6, 7, 8, 9, 10, 11, 12, 13, 14, 15, 16, 17, 18, 19, 20, 21, 22, or 23 amino acids at its N-terminus and/or at its C-terminus and/or internally as compared to the parent polypeptide, preferably at its N-terminus, at its N- and C-terminus, or at its C-terminus. Ebenhoch R. et al., 2019 suggest that the receptor interaction is more likely to take place in the front/protruding part of the top face of MYDGF and indicates that Tyr73 may be the key residue for receptor interaction.

In case where two sequences are compared and the reference sequence is not specified in comparison to which the sequence identity percentage is to be calculated, the sequence identity is to be calculated with reference to the longer of the two sequences to be compared, if not specifically indicated otherwise.

The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90:5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) or the CLUSTALW2 algorithm (Larkin M A, Blackshields G, Brown N P, Chenna R, McGettigan P A, McWilliam H, Valentin F, Wallace I M, Wilm A, Lopez R, Thompson J D, Gibson T J, Higgins D G. (2007). Clustal W and Clustal X version 2.0.Bioinformatics, 23, 2947-2948.) which are available e.g. on http://npsa-pbil.ibcp.fr/cgi-bin/npsa_automat.pl?page=/NPSA/npsa_clustalw.html or on http://www.ebi.ac.uk/Tools/clustalw2/index.html. Preferably, the CLUSTALW2 algorithm on http://www.ebi.ac.uk/Tools/clustalw2/index.html is used wherein the parameters used are the default parameters as they are set on http://www.ebi.ac.uk/Tools/clustalw2/index.html: Alignment type=Slow, protein weight matrix=Gonnet, gap open=10, gap extension=0.1 for slow pairwise alignment options and protein weight matrix=Gonnet, gap open=10, gap extension=0,20, gap distances=5, No end gaps=no, Output options: format=Aln w/numbers, Order=aligned.

The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215:403-410. BLAST protein searches are performed with the BLASTP program available e.g. on http://blast.ncbi.nlm.nih.gov/Blast.cgi?PROGRAM=blastp&BLAST_PROGRAMS=blastp&P AGE_TYPE=BlastSearch&SHOW_DEFAULTS-on&LINK_LOC-blasthome. Preferred algorithm parameters used are the default parameters as they are set on http://blast.ncbi.nlm.nih.gov/Blast.cgi?PROGRAM=blastp&BLAST_PROGRAMS=blastp&P AGE_TYPE=BlastSearch&SHOW_DEFAULTS=on&LINK_LOC=blasthome: Expect threshold=10, word size=3, max matches in a query range=0, matrix=BLOSUM62, gap costs=Existence: 11 Extension: 1, compositional adjustments=conditional compositional score matrix adjustment together with the database of non-redundant protein sequences (nr) to obtain amino acid sequences homologous to the Factor 1 and Factor 2 polypeptides.

To obtain gapped alignments for comparative purposes, Gapped BLAST is utilized as described in Altschul et al. (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise.

The term “host cell” as used herein refers to a cell that harbours a nucleic acid of the invention (e.g. in form of a plasmid or virus). Such host cell may either be a prokaryotic (e.g. a bacterial cell) or a eukaryotic cell (e.g. a fungal, plant or animal cell). The cell can be transformed or non-transformed. The cell can be an isolated cell for example in a cell culture or part of a tissue, which itself can be isolated or part of a more complex organization structure such as an organ or an individual.

The terms “myeloid-derived growth factor”, “MYDGF”, “Factor 1”, “MYDGF polypeptide or protein” or “Factor 1 polypeptide or protein” are used interchangeably and refer to the protein indicated in NCBI reference sequence NM_019107.3 (human homologue) as well as to its mammalian homologues, in particular from mouse or rat. The amino acid sequence of the human homologue is encoded in open reading frame 10 on human chromosome 19 (C19Orf10). Preferably, MYDGF and Factor 1 protein refer to a protein, which comprises, essentially consists or consists of a core segment of human Factor 1 having an amino acid sequence according to SEQ ID NO: 1.

According to a further preferred embodiment, MYDGF and Factor 1 protein, respectively, refer to a protein, which comprises SEQ ID NO: 3. In a further preferred embodiment, MYDGF refers to a protein, which essentially consists of SEQ ID NO: 3. In a further preferred embodiment, MYDGF refers to a protein, which consists of SEQ ID NO: 3.

As used herein, MYDGF as defined herein and fragments and variants thereof include pharmaceutically acceptable salts thereof.

Whether or not a protein, variant or fragment exhibits the biological function of MYDGF can be determined by any one of the tests described in the examples below. According to the present invention, a peptide or protein exhibits the biological function of MYDGF if the results obtained with such peptide or protein compared to the results obtained with the MYDGF protein of the present invention shown in at least one of the examples presented herein below achieve at least 50%, at least 60%, at least 70%, at least 80%, at least 90%, at least 95% or 100% of the effect reported for MYDGF over the indicated controls.

As used herein, the terms “MYDGF” and “Mydgf” both denote myeloid-derived growth factor.

The term “cardiogenic shock” describes a state of end-organ hypoperfusion due to cardiac failure and the inability of the cardiovascular system to provide adequate blood flow to the extremities and vital organs. Patients with cardiogenic shock manifest persistent hypotension (systolic blood pressure less than 80 to 90 mm Hg, or a mean arterial pressure 30 mm Hg below baseline, or vasopressor or inotropes support to maintain SBP >90 mm Hg, or mean arterial blood pressure <70 mm Hg, or systolic blood pressure <100 mm Hg despite adequate fluid resuscitation), with evidence of end-organ damage (as evidenced for example by urine output <30 mL/h, or urine output <0.5 mL/kg for 1 h, or cool extremities, or mottled skin, or serum lactate >2 mmol/L, or metabolic acidosis, or altered mental status), with a severe reduction in cardiac index (less than 2.2 L/min per m²) in the presence of adequate or elevated filling pressure (left ventricular [LV] end-diastolic pressure above 15 mm Hg or right ventricular (RV) end-diastolic pressure above 10 to 15 mm Hg) (Vahdatpour et al., Journal of the American Heart Association, Vol 8 (8), 2019, e011991).

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that have previously had the disorder(s); (e) limiting or preventing recurrence of symptoms in individuals that were previously symptomatic for the disorder(s); (f) reduction of mortality after occurrence of a disease or a disorder; (g) healing; and (h) prophylaxis of a disease. The term “ameliorating” is also encompassed by the term “treating”. In line therewith, the term “treating and/or preventing” a condition or disease as mentioned herein means that the condition or disease is treated or prevented, or both.

The term “stroke volume” describes the volume of blood ejected by the right/left ventricle in a single contraction. It is the difference between the end-diastolic volume (EDV) and the end-systolic volume (ESV).

The term “stroke work” describes the work performed by the left or right ventricle to eject the stroke volume into the aorta or pulmonary artery, respectively.

The term “cardiac output” describes the amount of blood pumped by the ventricle in unit time.

The terms “dP/dtₘᵢₙ” and “dP/dtₘₐₓ” describe the minimum and maximum rate of pressure change in the ventricle, respectively. Peak dP/dt is used as an index of ventricular performance.

The term “isovolumic relaxation constant” or “Tau” describes the exponential decay of the ventricular pressure during isovolumic relaxation. It is also referred to as the ventricular diastolic time constant.

The term “fraction of inhaled oxygen” or “FiO₂” describes the molar or volumetric fraction of oxygen in the inhaled gas. Natural air includes 21% oxygen, which is equivalent to FiO₂ of 0.21.

The terms “subject”, “individual” and “patient” are used herein interchangeable and refer to an individual, such as a human, a non-human primate (e.g. chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. In a particular meaning, the subject is a mammal. In a preferred meaning, the subject is a human. The subject can be a healthy subject or a subject suffering from or suspected of having one or more diseases. A subject suffering from or suspected of having one or more diseases is also referred to as a patient.

These descriptions and definitions are valid for the whole application unless it is otherwise stated.

Sequences
Sequences used in the present invention are listed
below.
SEQ ID NO: 1 (amino acid sequence of human
Factor 1 (MYDGF), lacking the 31 aa
N-terminal signal peptide):
VSEPTTVAFDVRPGGVVHSFSHNVGPGDKYTCMFTYASQGGTNEQWQMSL
GTSEDHQHFTCTIWRPQGKSYLYFTQFKAEVRGAEIEYAMAYSKAAFERE
SDVPLKTEEFEVTKTAVAHRPGAFKAELSKLVIVAKASRTEL
SEQ ID NO: 2 (amino acid sequence of MYDGF,
including the N-terminal signal peptide (shown in
bold and underlined); UniProtKB - Q969H8):
MAAPSGGWNGVGASLWAALLLGAVALRPAEAVSEPTTVAFDVRPGGVVH
SFSHNVGPGDKYTCMFTYASQGGTNEQWQMSLGTSEDHQHFTCTIWRPQ
GKSYLYFTQFKAEVRGAEIEYAMAYSKAAFERESDVPLKTEEFEVTKTA
VAHRPGAFKAELSKLVIVAKASRTEL
SEQ ID NO: 3 (amino acid sequence of the [+G]
MYDGF variant, in which the N-terminal V residue
in position +1 of the mature human MYDGF is
preceded by an G residue):
GVSEPTTVAFDVRPGGVVHSFSHNVGPGDKYTCMFTYASQGGTNEQWQMS
LGTSEDHQHFTCTIWRPQGKSYLYFTQFKAEVRGAEIEYAMAYSKAAFER
ESDVPLKTEEFEVTKTAVAHRPGAFKAELSKLVIVAKASRTEL
SEQ ID NO: 4 shows the nucleic acid sequence of
human Factor 1 encoding MYDGF
of SEQ ID NO: 3 (NCBI Gene ID: 56005).

### EMBODIMENTS

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The present disclosure shows for the first time anti-cardiogenic shock effects for MYDGF. The disclosure particularly shows that administration of MYDGF in a mouse model of cardiogenic shock attenuates symptoms associated with cardiogenic shock. Specifically, the disclosure shows that treatment of mice of an animal model of cardiogenic shock with MYDGF increases cardiac output, stroke work, stroke volume, ventricular end-systolic pressure and ventricular ejection fraction compared to control mice treated with saline. The disclosure further shows that MYDGF successfully treats cardiogenic shock and increases overall survival (cf. examples below).

Therefore, in a first aspect, the invention provides the protein myeloid-derived growth factor (MYDGF) or a fragment or a variant thereof for use in treating and/or preventing cardiogenic shock. According to a preferred embodiment, the fragment or variant of MYDGF exhibits the biological function of MYDGF.

According to a preferred embodiment, the cardiogenic shock to be treated or prevented is in the course of (acute) myocardial infarction (AMI, MI), preferably ST elevation myocardial infarction (STEMI), even more preferably a cardiogenic shock in the course of a MI or STEMI in the course of reperfusion following Percutaneous Coronary Intervention (PCI).

In a particularly preferred embodiment of the invention, the protein comprises the amino acid sequence SEQ ID NO: 1 or a fragment thereof. Preferably, the protein has at least 85%, 86%, 87%, 88%, 89%, 90%, 91%, 92%, 93%, 94%, 95%, 96%, 97%, 98%, or 99% identity to SEQ ID NO: 1.

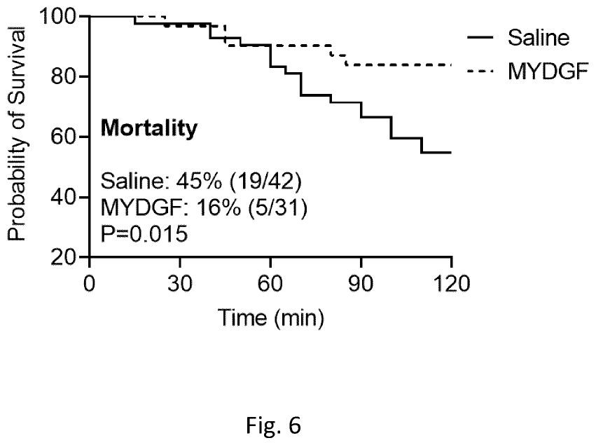

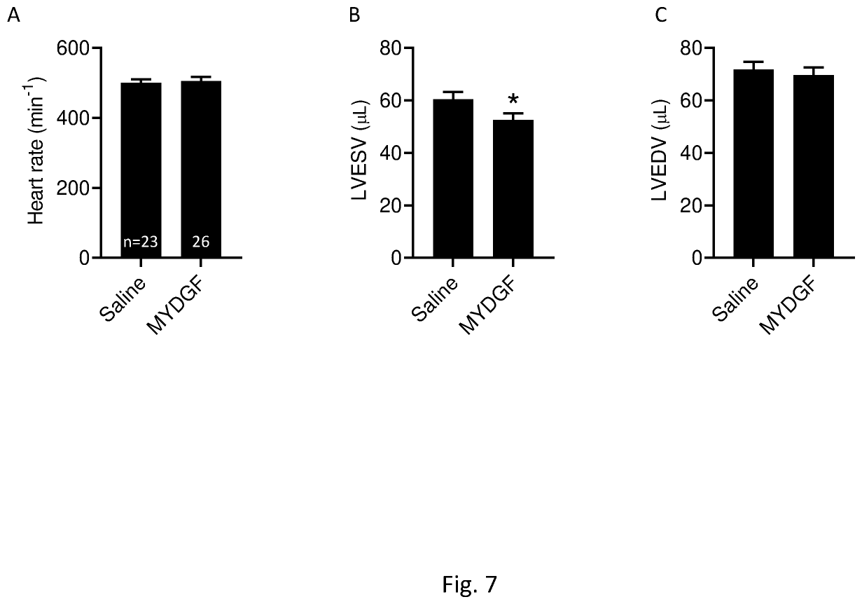

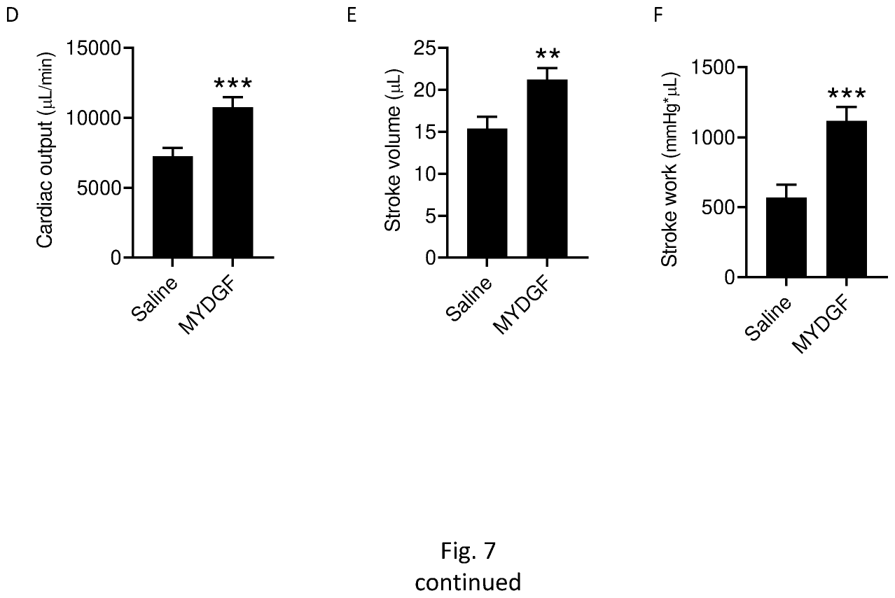

In a preferred embodiment of this aspect of the invention, the protein comprises the amino acid sequence SEQ ID NO: 1, a fragment or a variant thereof which has at least 85% sequence identity to SEQ ID NO: 1. According to a preferred embodiment, the fragment or variant of MYDGF exhibits the biological function of MYDGF. A person skilled in the art is able to decide without undue burden, which positions in the parental polypeptide can be mutated to which extent and which positions have to be maintained to preserve the functionality of the polypeptide. Such information can, for example, be gained from homologues sequences which can be identified, aligned and analyzed by bioinformatic methods well known in the art. Such analyses are exemplarily described in example 7 and FIGS. 6 and 7 of WO 2014/111458. Mutations are preferably introduced in those regions of the protein, which are not fully conserved between species, preferably mammals. In a particularly preferred embodiment of the invention, the MYDGF protein comprises, essentially consists or consists of the amino acid sequence SEQ ID NO: 1 or 3, or a fragment or variant thereof. According to a preferred embodiment, the fragment or variant of MYDGF exhibits the biological function of MYDGF. Preferably, the protein has at least 85%, 86%, 87%, 88%, 89%, 90%, 91%, 92%, 93%, 94%, 95%, 96%, 97%, 98%, or 99% sequence identity to SEQ ID NO: 1.

N-terminal deletion variants are also encompassed, which may for example lack one or more amino acids from amino acid position 1 to 24 (based on SEQ ID NO: 1), i.e. from the N-terminally conserved region.

C-terminal deletion variants are also encompassed, which may for example lack one or more amino acids from amino acid position 114 to 142 (based on SEQ ID NO: 1).

On the other hand, amino acids can be added to the MYDGF protein. Such additions include additions at the N-terminus, at the C-terminus, within the amino acid sequence or combinations thereof. The protein of the first aspect of the present invention may thus further comprise additional amino acid sequences, e.g. for stabilizing or purifying the resulting protein. Examples of such amino acids are 6xHis-tags, myc-tags, or FLAG-tags, which are well known in the art, and which may be present at any position in the protein, preferably at the N-terminus or the C-terminus. A particularly preferred additional sequence is a 6xHis-tag. Preferably, said 6xHis-tag is present on the C-terminus of the MYDGF protein. Depending on the expression system used and, if present, an additional amino acid such as a tag described above, one or more residual amino acids may remain on the N-terminus and/or the C-terminus of the protein. It is emphasized that in the MYDGF protein and Mydgf protein according to the present invention such artefacts may be present, as in shown e.g. in Ebenhoch R. et al., Nat Commun. 2019 Nov. 26;10(1): 5379, and Polten F. et al., Anal Chem. 2019 Jan. 15;91(2): 1302-1308.

In some cases it is preferred to mutate protease cleavage sites within the MYDGF protein of the first aspect of the present invention to stabilize the protein (see Segers et al. Circulation 2007, 2011). The skilled person knows how to determine potential proteolytic cleavage sites within a protein. For example, protein sequences can be submitted to websites providing such analysis as, e.g. http://web.expasy.org/peptide_cutter/or http://pmap.burnham.org/proteases. If the protein sequence according to SEQ ID NO: 1 is submitted to http://web.expasy.org/peptide_cutter/the following cleavage sites with lower frequency (less than 10) are determined:

TABLE 1

| | | Position (with reference |
|---|---|---|
| Protease | Frequency | to SEQ ID NO: |
| Arg-C proteinase | 6 | 12 65 82 99 120 139 |
| Asp-N endopeptidase | 4 | 9 27 54 101 |
| Clostripain | 6 | 12 65 82 99 120 139 |
| LysN | 9 | 28 68 77 93 105 113 124 129 135 |
| Proline-endopeptidase | 3 | 13 66 121 |

These sites may be altered to remove the recognition/cleavage sequence of the respectively identified protease to increase the serum half-life of the protein.

The MYDGF protein may further comprise additional amino acid sequences, e.g. for stabilizing or purifying the resulting protein. For example, it is preferred to mutate protease cleavage sites within the MYDGF protein to stabilize the protein. Suitable proteolytic cleavage sites can be identified as described above.

The MYDGF protein or compositions comprising the protein can administered in vivo, ex vivo or in vitro, preferably in vivo.

Nucleic acid sequences can be optimized in an effort to enhance expression in a host cell. Parameters to be considered include C: G content, preferred codons, and the avoidance of inhibitory secondary structure. These Factors can be combined in different ways in an attempt to obtain nucleic acid sequences having enhanced expression in a particular host (cf. e.g. Donnelly et al., International Publication Number WO 97/47358). The ability of a particular sequence to have enhanced expression in a particular host involves some empirical experimentation. Such experimentation involves measuring expression of a prospective nucleic acid sequence and, if needed, altering the sequence. Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. The translation of a particular codon into a particular amino acid is well known in the art (see, e.g., Lewin GENES IV, p. 119, Oxford University Press, 1990). The present invention thus also provides a nucleic acid encoding the MYDGF or the fragment or variant thereof, for use in treating and/or preventing cardiogenic shock. According to a preferred embodiment, the fragment or variant of MYDGF exhibits the biological function of MYDGF. According to a further preferred embodiment, the nucleic acid encodes an amino acid sequence having at least 85% sequence identity to SEQ ID NO:1.

The nucleic acid for use according to the present invention may further comprise a transcriptional control element or expression control sequences positioned to control expression of the protein. Such a nucleic acid together with control elements is often termed as an expression system. The term “expression system” as used herein refers to a system designed to produce one or more gene products of interest. Typically, such system is designed “artificially”, i.e. by gene-technological means usable to produce the gene product of interest in vivo, in vitro or ex vivo. The term “expression system” further encompasses the expression of the gene product of interest comprising the transcription of the polynucleotides, mRNA splicing, translation into a polypeptide, co- and post-translational modification of a polypeptide or protein as well as the targeting of the protein to one or more compartments inside of the cell, the secretion from the cell and the uptake of the protein in the same or another cell. This general description refers to expression systems for the use in eukaryotic cells, tissues or organisms. Expression systems for prokaryotic systems may differ, wherein it is well known in the art, how an expression system for prokaryotic cells is constructed.

Regulatory elements present in a gene expression cassette generally include: (a) a promoter transcriptionally coupled to a nucleotide sequence encoding the polypeptide, (b) a 5′ ribosome binding site functionally coupled to the nucleotide sequence, (c) a terminator joined to the 3′ end of the nucleotide sequence, and (d) a 3′ polyadenylation signal functionally coupled to the nucleotide sequence. Additional regulatory elements useful for enhancing or regulating gene expression or polypeptide processing may also be present. Promoters are genetic elements that are recognized by an RNA polymerase and mediate transcription of downstream regions. Preferred promoters are strong promoters that provide for increased levels of transcription. Examples of strong promoters are the immediate early human cytomegalovirus promoter (CMV), and CMV with intron A (Chapman et al, Nucl. Acids Res. 19:3979-3986, 1991). Additional examples of promoters include naturally occurring promoters such as the EF1 alpha promoter, the murine CMV promoter, Rous sarcoma virus promoter, and SV40 early/late promoters and the [beta]-actin promoter; and artificial promoters such as a synthetic muscle specific promoter and a chimeric muscle-specific/CMV promoter (Li et al., Nat. Biotechnol. 17:241-245, 1999, Hagstrom et al., Blood 95:2536-2542, 2000).

The ribosome binding site is located at or near the initiation codon. Examples of preferred ribosome binding sites include CCACCAUGG, CCGCCAUGG, and ACCAUGG, where AUG is the initiation codon (Kozak, Cell 44:283-292, 1986). The polyadenylation signal is responsible for cleaving the transcribed RNA and the addition of a poly(A) tail to the RNA. The polyadenylation signal in higher eukaryotes contains an AAUAAA sequence about 11-30 nucleotides from the polyadenylation addition site. The AAUAAA sequence is involved in signaling RNA cleavage (Lewin, Genes IV, Oxford University Press, NY, 1990). The poly (A) tail is important for the processing, export from the nucleus, translation and stability of the mRNA.

Polyadenylation signals that can be used as part of a gene expression cassette include the minimal rabbit [beta]-globin polyadenylation signal and the bovine growth hormone polyadenylation (BGH) (Xu et al., Gene 272:149-156, 2001, Post et al., U.S. Pat. No. 5,122,458).

Examples of additional regulatory elements useful for enhancing or regulating gene expression or polypeptide processing that may be present include an enhancer, a leader sequence and an operator. An enhancer region increases transcription. Examples of enhancer regions include the CMV enhancer and the SV40 enhancer (Hitt et al., Methods in Molecular Genetics 7:13-30, 1995, Xu, et al., Gene 272:149-156, 2001). An enhancer region can be associated with a promoter.

The expression of the MYDGF protein or of the variant thereof according to the present invention may be regulated. Such regulation can be accomplished in many steps of the gene expression. Possible regulation steps are, for example but not limited to, initiation of transcription, promoter clearance, elongation of transcription, splicing, export from the nucleus, mRNA stability, initiation of translation, translational efficiency, elongation of translation and protein folding. Other regulation steps, which influence the concentration of a MYDGF polypeptide inside a cell affect the half-life of the protein. Such a regulation step is, for example, the regulated degeneration of proteins. As the proteins of the invention comprise secreted proteins, the protein can be directed to a secretory pathway of the host cell. The efficiency of secretion regulates together with the regulatory steps referring to the expression and protein stability the concentration of the respective protein outside of the cell. Outside of the cell can refer to, for example but not limited to, a culture medium, a tissue, intracellular matrix or space or a body fluid such as blood or lymph.

The control of the regulatory steps mentioned above can be, for example, cell-type or tissue-type independent or cell-type or tissue-type specific. In a particularly preferred embodiment of the invention, the control of the regulatory steps is cell-type or tissue-type specific. Such a cell-type or tissue-type specific regulation is preferably accomplished through the regulation steps referring to the transcription of a nucleic acid. This transcriptional regulation can be accomplished through the use of cell-type or tissue-type specific promoter sequences. The result of this cell-type or tissue-type specific regulation can have different grades of specificity. This means, that the expression of a respective polypeptide is enhanced in the respective cell or tissue in comparison to other cell- or tissue-type or that the expression is limited to the respective cell- or tissue-type. Cell- or tissue-type specific promoter sequences are well known in the art and available for a broad range of cell- or tissue-types.

The expression is not necessarily cell-type or tissue-type specific but may depend from physiological conditions. Such conditions are for example an inflammation or a wound. Such a physiological condition-specific expression can also be accomplished through regulation at all above mentioned regulation steps. The preferred way of regulation for a physiological condition-specific expression is the transcriptional regulation. For this purpose a wound or inflammation specific promoter can be used. Respective promoters are, for example, natural occurring sequences, which can be, for example, derived from genes, which are specifically expressed during an immune reaction and/or the regeneration of wounded tissue. Another possibility is the use of artificial promoter sequences, which are, for example constructed through combination of two or more naturally occurring sequences.

The regulation can be cell-type or tissue-type specific and physiological condition-specific. Particularly, the expression can be a heart specific expression. Preferably, the expression is heart specific and/or wound specific.

Another possibility for a regulation of expression of the MYDGF protein or variant thereof according to the present invention is the conditional regulation of the gene expression. To accomplish conditional regulation, an operator sequence can be used. For example, the Tet operator sequence can be used to repress gene expression. The conditional regulation of gene expression by means of the Tet operator together with a Tet repressor is well known in the art and many respective systems have been established for a broad range of prokaryotic and eukaryotic organisms. A person of skill in the art knows how to choose a suitable system and adapt it to the special needs of the respective application.

In a particularly preferred embodiment, the use of a nucleic acid according to the invention comprises the application to an individual or patient, preferably an individual or patient suffering from cardiogenic shock.

According to a further aspect, the present invention provides vectors comprising the nucleic acid or the expression system described herein for use in treating and/or preventing cardiogenic shock.

As used herein, the term “vector” refers to a protein or a polynucleotide or a mixture thereof which is capable of being introduced or of introducing the proteins and/or nucleic acid comprised therein into a cell. In the context of the present invention it is preferred that the genes of interest encoded by the introduced polynucleotide are expressed within the host cell upon introduction of the vector or the vectors. Examples of suitable vectors include but are not limited to plasmid vectors, cosmid vectors, phage vectors such as lambda phage, filamentous phage vectors, viral vectors, viral like particles, and bacterial spores.

In a preferred embodiment of the invention, the vector is a viral vector. Suitable viral vectors include but are not limited to adenoviral vectors, adeno-associated viral (AAV) vectors, alphaviral vectors, herpes viral vectors, measles viral vectors, pox viral vectors, vesicular stomatitis viral vectors, retroviral vector and lentiviral vectors.

In a particularly preferred embodiment of the invention, the vector is an adenoviral or an adeno-associated viral (AAV) vector.

Nucleic acids encoding one or more MYDGF proteins or variants thereof according to the invention can be introduced into a host cell, a tissue or an individual using vectors suitable for therapeutic administration. Suitable vectors can preferably deliver nucleic acids into a target cell without causing an unacceptable side effect.

In a particularly preferred embodiment the use of a vector according to the invention, comprises the application to an individual in need thereof.

Vectors comprising nucleic acids encoding the MYDGF protein or fragments or variants thereof preferably exhibiting the biological function of MYDGF described above are preferably for use in treating and/or preventing cardiogenic shock.

According to a further aspect, the present invention provides a host cell comprising the vector as described herein and expressing the nucleic acid encoding the MYDGF protein or a fragment or a variant thereof for use in treating and/or preventing cardiogenic shock. According to a preferred embodiment, the fragment or variant of MYDGF exhibits the biological function of MYDGF.

According to a further aspect, the present invention provides pharmaceutical compositions comprising the MYDGF protein or a fragment or a variant thereof and optionally a suitable pharmaceutical excipient, for use in treating and/or preventing cardiogenic shock. According to a preferred embodiment, the fragment or variant of MYDGF exhibits the biological function of MYDGF.

The term “suitable pharmaceutical excipient”, as used herein, refers to a pharmacologically inactive substance such as but not limited to a diluent, excipient, surfactants, stabilizers, physiological buffer solutions or vehicles with which the therapeutically active ingredient is administered. “Pharmaceutical excipients” are also called “pharmaceutical carriers” and can be liquid or solid. Liquid carriers include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin. In a preferred embodiment of the invention, the carrier is a suitable pharmaceutical excipient. Suitable pharmaceutical excipients comprise starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. Such suitable pharmaceutical excipients are preferably pharmaceutically acceptable.

The terms “pharmaceutical”, “medicament” and “drug” are used interchangeably herein referring to a substance and/or a combination of substances being used for the identification, prevention and/or treatment of a tissue status or disease.

“Pharmaceutically acceptable” means approved by a regulatory agency of the Federal or a state government or listed in the U.S. Pharmacopeia or other generally recognized pharmacopeia for use in animals, and more particularly in humans. The term “pharmaceutically acceptable salt” refers to a salt of the protein or peptide of the present invention. Suitable pharmaceutically acceptable salts include acid addition salts which may, for example, be formed by mixing a solution of the peptide of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the peptide carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)).

The term “active ingredient” refers to the substance in a pharmaceutical composition or formulation that is biologically active, i.e. that provides pharmaceutical value. A pharmaceutical composition may comprise one or more active ingredients which may act in conjunction with or independently of each other. The active ingredient can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as but not limited to those derived from sodium, potassium, ammonium, calcium, ferric hydroxides, isopropylamine, triethylamine, 2-ethylamino ethanol, histidine, procaine, and the like.

The terms “preparation” and “composition” are intended to include the formulation of the active compound with encapsulating material as a carrier providing a capsule in which the active component with or without other carriers, is surrounded by a carrier, which is thus in association with it.

The term “carrier”, as used herein, refers to a pharmacologically inactive substance such as but not limited to a diluent, excipient, or vehicle with which the therapeutically active ingredient is administered. Such pharmaceutical carriers can be liquid or solid. Liquid carriers include but are not limited to sterile liquids, such as saline solutions in water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The term “composition” is preferably intended to include the formulation of the active compound with e.g. encapsulating material as a carrier providing a capsule in which the active component with or without other carriers, is surrounded by a carrier, which is thus in association with the active compound.

According to one embodiment, the active ingredient is administered to a cell, a tissue or an individual in a therapeutically effective amount. A “therapeutically effective amount” is an amount of an active ingredient sufficient to achieve the intended purpose. The active ingredient may be a therapeutic agent. The effective amount of a given active ingredient will vary with parameters such as the nature of the ingredient, the route of administration, the size and species of the individual to receive the active ingredient, and the purpose of the administration. The effective amount in each individual case may be determined empirically by a skilled artisan according to established methods in the art. As used in the context of the invention, “administering” includes in vivo administration to an individual as well as administration directly to cells or tissue in vitro or ex vivo.

In a preferred embodiment of the invention, the pharmaceutical composition is customized for the treatment of a disorder and specifically for the treatment of cardiogenic shock. In a further preferred embodiment of the invention, the pharmaceutical composition is customized for the prevention of a disorder and specifically for the prevention of cardiogenic shock. According to a particularly preferred embodiment, the pharmaceutical composition is customized for the prevention and treatment of cardiogenic shock. In a particularly preferred embodiment of the invention, treatment with a pharmaceutical composition according to the invention comprises the treatment of an individual in need of such treatment and/or the prevention of cardiogenic shock in an individual in need thereof.

The pharmaceutical composition contemplated by the present invention may be formulated in various ways well known to one of skill in the art. For example, the pharmaceutical composition of the present invention may be in liquid form such as in the form of solutions, emulsions, or suspensions. Preferably, the pharmaceutical composition of the present invention is formulated for parenteral administration, preferably for intravenous, intra-arterial, intramuscular, subcutaneous, transdermal, intrapulmonary, intraperitoneal intracoronary, intra-cardiac administration, or administration via mucous membranes, preferably for intravenous, subcutaneous, or intraperitoneal administration. A preparation for oral or anal administration is also possible. Preferably, the pharmaceutical composition of the present invention is in the form of a sterile aqueous solution which may contain other substances, for example, enough salts or glucose to make the solution isotonic with blood. The aqueous solutions should be suitably buffered (preferably to a pH of from 3 to 9, more preferably to a pH of from 5 to 7), if necessary. The pharmaceutical composition is preferably in unit dosage form. In such form the pharmaceutical composition is subdivided into unit doses containing appropriate quantities of the active component. The unit dosage form can be a packaged preparation, the package containing discrete quantities of pharmaceutical composition such as vials or ampoules.

The pharmaceutical composition is preferably administered through the intravenous, intra-arterial, intramuscular, subcutaneous, transdermal, intrapulmonary, intraperitoneal, intracoronary or intra-cardiac route, wherein other routes of administration known in the art are also comprised.

If the pharmaceutical composition is used as a treatment for an individual or in the prophylaxis of a disease or disorder, the use of the pharmaceutical composition can replace the standard treatment or prophylaxis for the respective disease or condition or can be administered additionally to the standard treatment. In the case of an additional use of the pharmaceutical composition, the pharmaceutical composition can be administered before, simultaneously or after a standard therapy and/or prophylaxis.

It is further preferred that the pharmaceutical composition is administered once or more than once. This comprises 2, 3, 4, 5, 6, 7, 8, 9, 10, 11, 12, 13, 14, 15, 16, 17, 18, 19, 20, 25, 30, 35, 40, 45 or 50 times. The time span for the administration of the pharmaceutical is not limited. Preferably, the administration does not exceed 1, 2, 3, 4, 5, 6, 7 or 8 weeks.

A single dose of the pharmaceutical composition, can independently form the overall amount of administered doses, or the respective time span of administration can include administration as one or more bolus injection(s) and/or infusion(s).

According to a further aspect, the present invention provides a method of treating and/or preventing cardiogenic shock, comprising administering to a patient in need thereof a therapeutically effective amount of MYDGF or fragment or variant thereof. Suitable MYDGF proteins, fragments, or variants thereof include those described for the first aspect. According to a preferred embodiment, the fragment or variant of MYDGF exhibits the biological function of MYDGF. In said method, the MYDGF preferably comprises SEQ ID NO: 1, or a fragment or variant thereof exhibiting the biological function of MYDGF of SEQ ID NO: 1. In this respect, the fragment or variant preferably comprises an amino acid sequence with at least 85% amino acid sequence identity to SEQ ID NO:1.

Administering may be carried out, for example, as described for the first aspect. According to a preferred embodiment of this method of the present invention, the MYDGF protein or fragment or variant thereof is administered through one or more bolus injection(s) and/or infusion(s), preferably in a pharmaceutically accepted carrier and/or excipient.

According to a further aspect, the present invention provides a method for producing a non-human mammalian model of cardiogenic shock, the method comprising (i) transiently ligating a coronary artery of the mammal, (ii) establishing reperfusion, and (iii) ventilating the mammal with a fraction of inhaled oxygen (FiO₂) of about 0.18 or less. Transient ligation can be performed by any method known to the skilled person as being suitable for blocking or significantly reducing blood flow. According to a preferred embodiment, ligation is performed by placing and tightening a surgical thread or surgical wire around a coronary artery, thereby blocking or essentially blocking blood flow through the artery. Reperfusion is preferably established by reestablishing blood flow, e.g. by opening the surgical thread or surgical wire, allowing blood to flow through the artery. Ventilation of the mammal is performed by standard means known in the art, e.g. mechanical ventilation commonly used in surgery of mammals.

This method specifically does not provide a treatment to the animal but rather induces a condition that resembles cardiogenic shock. Cardiogenic shock is characterized for example by a lowered ventricular end-systolic pressure (VESP) and reduced cardiac output as well as a raised arterial lactate concentration compared to a healthy subject or compared to the subject before the method has been carried out. The novel non-human mammalian model of cardiogenic shock has the advantage that small animals such as rodents can be used, that are established in laboratory practice. There is no necessity for using larger animals such as pigs, which involve higher costs for food, shelter and animal husbandry, and which are more complicated to investigate. The new model allows e.g. genetically modified mice to be investigated, for example to explore molecular mechanisms of cardiogenic shock. Genetically modified mice can be easily generated, or are already available in the research community, or can be commercially obtained e.g. from The Jackson Laboratory. In contrast, genetically modified larger animals such as pigs are extremely difficult to generate and only few genetically modified large animals are available.

Therefore, according to a preferred embodiment, the non-human mammal is a rodent. According to a particularly preferred embodiment, the non-human animal is a mouse.

According to a preferred embodiment, the coronary artery is the proximal left anterior descending coronary artery. Alternatively, any other major coronary heart artery can be used which transient ligation eventually results in reduced ventricular end-systolic pressure (VESP) and increased arterial lactate concentration.

According to a further preferred embodiment, the coronary artery is ligated for about 30 minutes to about 90 minutes before reperfusion is established. According to a further preferred embodiment, the coronary artery is ligated for a time selected from a range having a lower value of about 30, 35, 40, 45, 50, 55, 60, 65, 70, 75, 80, or 85 minutes and an upper value of about 35, 40, 45, 50, 55, 60, 65, 70, 75, 80, 85, or 90 minutes, or in any combination thereof. More preferably, the coronary artery is ligated for about 45 to 70 minutes, and most preferably for about 60 minutes.

According to a further preferred embodiment, the mammal is ventilated with a fraction of inhaled oxygen (FiO₂) of between about 0.18 and about 0.12. According to a further preferred embodiment, the mammal is ventilated with a fraction of inhaled oxygen of an amount selected from a range having a lower value of about 0.12, 0.13, 0.14, 0.15, 0.16, or 0.17 and an upper value of about 0.13, 0.14, 0.15, 0.16, 0.17, or 0.18, and in any combination thereof. More preferably, the mammal is ventilated with a fraction of inhaled oxygen of between about 0.17 and about 0.14, most preferably of about 0.16.

The present invention also provides model animals obtained by performing the method for producing a non-human mammalian model of cardiogenic shock.

### EXAMPLES

The Examples are designed to further illustrate the present invention and serve a better understanding. They are not to be construed as limiting the scope of the invention in any way.

Materials and methods used in the Examples:

Unless stated otherwise, the following materials and methods were used in the examples.

### Recombinant MYDGF.

In the [+G] MYDGF variant (HEK) used in the examples, the N-terminal V residue in position+1 of the mature human MYDGF is preceded by a G residue. The variant has the sequence as denoted in SEQ ID NO: 3 and was manufactured as described in Polten, F. et al. (2019), Anal Chem, 91, 1302-1308 on page 1303, 1^{st} column and Figure S1, and in Ebenhoch, R. et al. (2019), Nat Commun 10, 5379 on page 8, left column.

### Mouse Surgery and Functional Assessment.

All surgical procedures were approved by the authorities in Hannover, Germany (Niedersächsisches Landesamt für Verbraucherschutz und Lebensmittelsicherheit). All animal procedures conformed to the guidelines from the EU directive 2010/63 on the protection of animals used for scientific purposes. Mice were housed in individually ventilated cages on a 12-hour light/dark cycle in the central animal facility of Hannover Medical School. Food and water were provided ad libitum. During surgery, mice were placed on a heating pad connected to a temperature controller (Föhr Medical Instruments) to keep rectal temperature at 37° C.

### Statistical Analyses.

Mouse littermates were randomly allocated to the different experimental groups. Based on visual inspection, data were normally distributed with similar variances in the different groups. Data are presented as mean±s.e.m . . . . The 2-independent-sample t test was used to compare 2 groups. For comparisons among more than 2 groups, 1-way ANOVA was used if there was one independent variable and 2-way ANOVA if there were two independent variables. Dunnett's post hoc test was used for multiple comparisons with a single control group. Tukey's post hoc test was used to adjust for multiple comparisons. A 2-tailed P value less than 0.05 was considered to indicate statistical significance. K.C.W. had full access to all data in the study and takes responsibility for the integrity of the data and the data analysis.

### Example 1

Establishing an animal model of cardiogenic shock. Myocardial infarction (MI) was induced in 8 to 10-week-old male C57BL/6N mice by transient proximal left anterior descending coronary artery ligation for 60 min. Animals were subcutaneously pretreated with 2 mg/kg butorphanol and with 0.02 mg/kg atropine to reduce bronchial secretions. Anaesthesia was induced with 3 to 4% isoflurane. After intubation, mice were mechanically ventilated and anaesthesia was maintained with 1 to 2% isoflurane. After a left thoracotomy, the left anterior descending coronary artery (LAD) was ligated with a 7-0 prolene (Ethicon, cat no. EH7405) slipknot to induce ischemia, which was removed 1 h later to induce reperfusion. In sham-operated mice, the ligature around the LAD was not tied. After coronary reperfusion, a micromanometer-tipped conductance catheter was inserted via the right carotid artery to record left ventricular (LV) pressure-volume loops. At the end of the protocol, an arterial blood sample was drawn from the left ventricle.

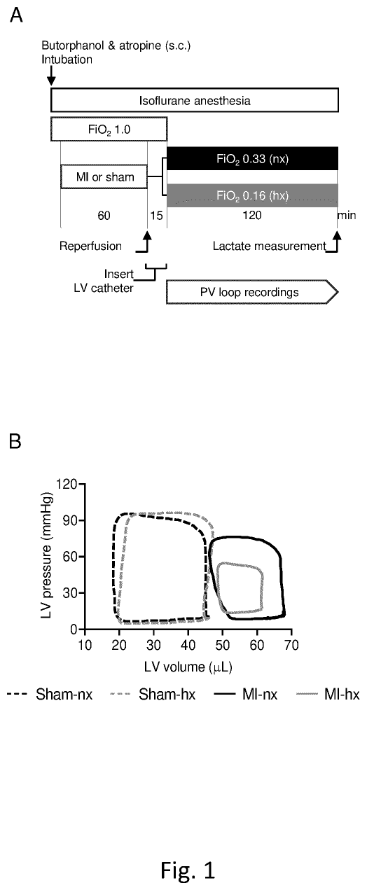

In pilot experiments, coronary artery ligation alone only slightly lowered LV end-systolic pressure (LVESP) and did not raise arterial lactate concentration, a biomarker of peripheral hypoperfusion and tissue hypoxia in cardiogenic shock (CS). Based on the premise that MI combined with hypoxic ventilation might induce CS, different fractions of inspired oxygen (FiO₂) were compared for ventilating mice after reperfusion (medical O₂ was mixed with medical N₂). During ongoing isoflurane anaesthesia, infarcted and sham operated mice were randomly ventilated with an FiO₂ of 0.33 associated with normoxaemia (nx; arterial oxygen partial pressure [PaO₂], 144±16 mm Hg; arterial oxygen saturation [SaO₂], 99±1%), or an FiO₂ of 0.16 resulting in mild hypoxaemia (hx; PaO₂, 75±16 mmHg; SaO₂, 89±3%; 4 to 6 mice per group) (FIG. 1A).

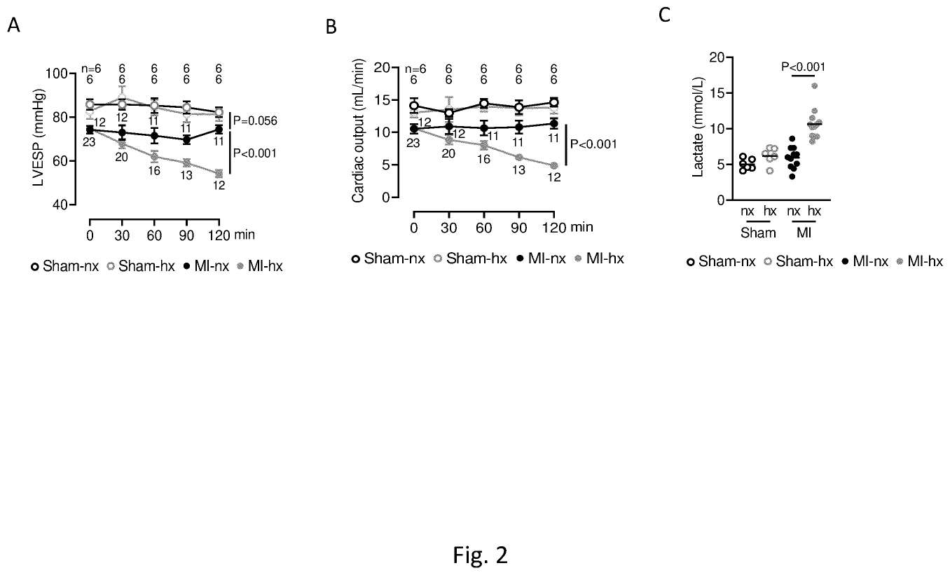

Exemplary pressure volume loops recorded after 120 min are presented (FIG. 1B). During the 120 min time period, LVESP (FIG. 2A) and cardiac output (FIG. 2B) gradually declined in MI-hx mice (MI with CS) but remained stable in MI-nx mice (MI without CS). At 120 min, MI-hx mice had developed arterial hyperlactataemia (FIG. 2C) and displayed more severe systolic and diastolic dysfunction than MI-nx mice (LV ejection fraction, 18±2 vs 33±2%, P<0.001; LV end-diastolic pressure, 14±1 vs 9±1 mmHg, P=0.005). Heart rate was not affected (475±16 vs 486±8 min-1). Mortality was higher in MI-hx mice (11 of 23 mice died; all deaths were related to CS) than in MI-nx mice (1 of 12 mice died, P=0.031).

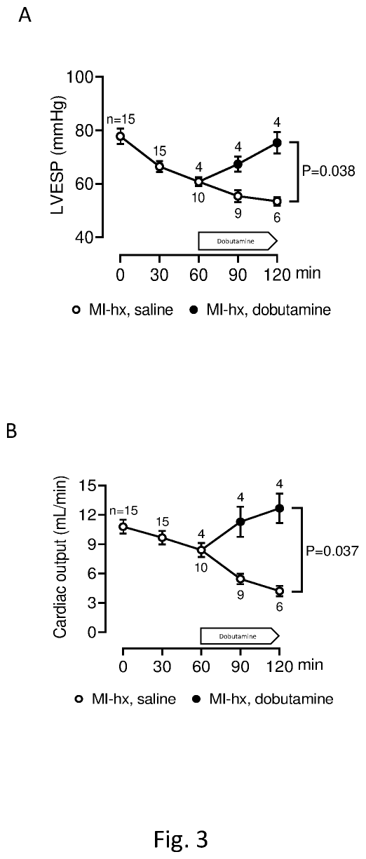

Intravenous dobutamine infusion, an established symptomatic therapy for patients with CS (Vahdatpour et al. Journal of the American Heart Association, Vol 8 (8), 2019, e011991), increased LVESP (FIG. 3A) and cardiac output (FIG. 3B), and lowered arterial lactate (5.6±0.6 vs 11.1±0.7 mmol/L, P<0.001, 4-6 mice per group) in MI-hx mice.

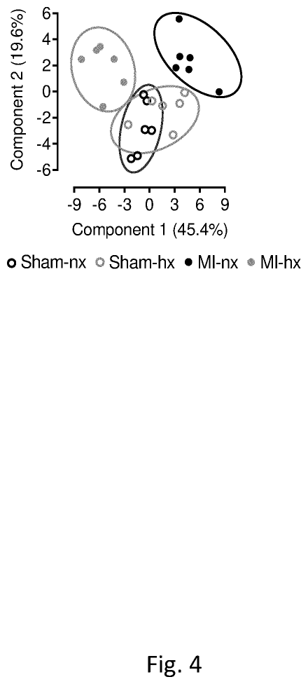

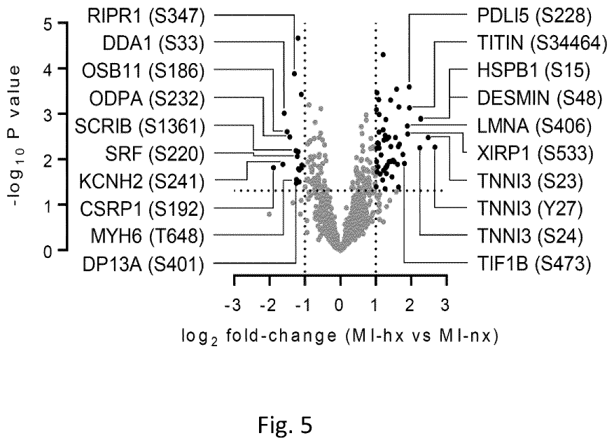

As a use case for the model, high-resolution mass spectrometry was employed to define phosphoproteome signatures in the non-infarcted LV myocardium at 120 min. Among 1,264 different proteins, 9,004 phosphosites were detected. Principle component analysis indicated that the four groups (Sham-nx=sham operated mice normoxaemia; Sham-hx=sham operated mice hypoxaemia; MI-nx=infarcted mice normoxaemia; MI-hx=infarcted mice hypoxaemia) were associated with distinct phosphoproteome signatures (FIG. 4). Reflecting the observation that hypoxaemia, in itself, did not alter cardiac performance (FIGS. 2A and 2B), the phosphoproteome signatures of sham-hx and sham-nx mice overlapped (FIG. 4). Conversely, MI-hx and MI-nx mice displayed vastly different phosphoproteome signatures (FIG. 4) with 72 differentially regulated phosphosites (FIG. 5), thus indicating potential therapeutic entry points.

A further detailed description is provided in Wang Y, Polten F, Jäckle F, Korf-Klingebiel M, Kempf T, Bauersachs J, Freitag-Wolf S, Lichtinghagen R, Pich A, Wollert K C. A mouse model of cardiogenic shock. Cardiovasc Res. 2021; 117:2414-2415, incorporated herein by reference in its entirety. The animal model of the present invention being the first of its kind, replicates key features of CS in patients, including severe systolic and diastolic dysfunction, low cardiac output and hypotension, increased arterial lactate concentration, hemodynamic response to dobutamine, and high mortality. The model provides a platform to explore molecular pathophysiology and develop desperately needed therapies for CS.

### Example 2

The MYDGF protein (human Factor 1; C19orf10) was identified as detailed in WO 2014/111458. The nucleic acid sequence encoding human Factor 1 is available under NCBI Gene ID: 56005 (SEQ ID NO: 4). The amino acid sequence of human Factor 1 including the N-terminal signal peptide is detailed in SEQ ID NO: 2. In the examples, the human [+G] MYDGF variant according to SEQ ID NO: 3 without the signal peptide was used and expressed as detailed in Ebenhoch R. et al., Crystal structure and receptor-interacting residues of MYDGF a protein mediating ischemic tissue repair (Nat Commun. 2019 Nov. 26;10(1):5379 and Polten et al. Plasma Concentrations of Myeloid-Derived Growth Factor in Healthy Individuals and Patients with Acute Myocardial Infarction as Assessed by Multiple Reaction Monitoring-Mass Spectrometry. Anal Chem. 2019 Jan. 15;91(2):1302-1308).

### Example 3

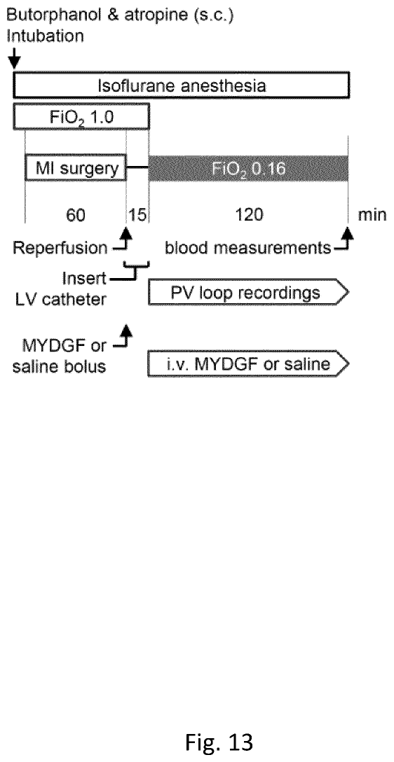

Acute myocardial infarction was induced in C57BL6/N mice, pretreated with butorphanol (2 mg/kg, subcutaneously (s.c.)) and atropine (0.02 mg/kg, s.c.) to reduce bronchial secretions, by transient left anterior descending coronary artery ligation for 60 minutes. Human MYDGF (10 μg in 100 μL) or saline (control) were bolus-injected into the left ventricular cavity at the time of reperfusion. After reperfusion, a micromanometer-tipped conductance catheter was inserted via the right carotid artery to continuously record left ventricular pressure-volume (PV) loops. Thereafter, cardiogenic shock (CS) was induced by hypoxic ventilation (FiO₂ 0.16) and mice received a continuous intra-left jugular vein infusion of either MYDGF (5 μg/h, infusion rate: 2 μL/min) or saline (control) for 120 minutes. After 120 min, an arterial blood sample (anticoagulated with heparin) was drawn from the left ventricle and used for immediate blood gas and lactate analysis. Another blood sample was withdrawn, treated with EDTA, and centrifuged at 3,500 g for 10 min at 4° C. to obtain plasma which was stored at −80° C. until further analysis. FIG. 13 schematically illustrates this process (abbreviations in FIG. 13 as defined for FIG. 1; s.c.=subcutaneous administration; i.v.=intravenous administration).

Results are shown in FIGS. 6 to 12.

MYDGF significantly improved the survival rate of cardiogenic shock mice as shown in FIG. 6.

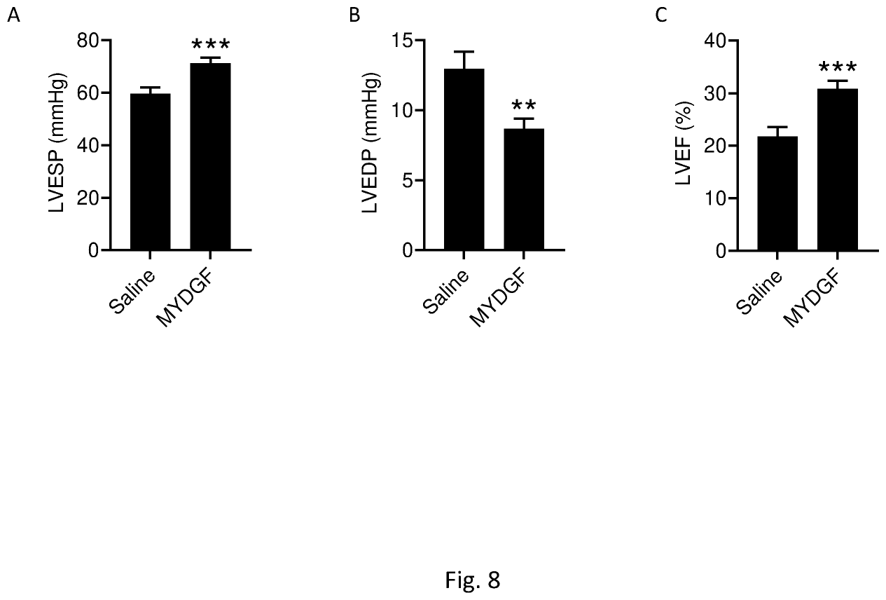

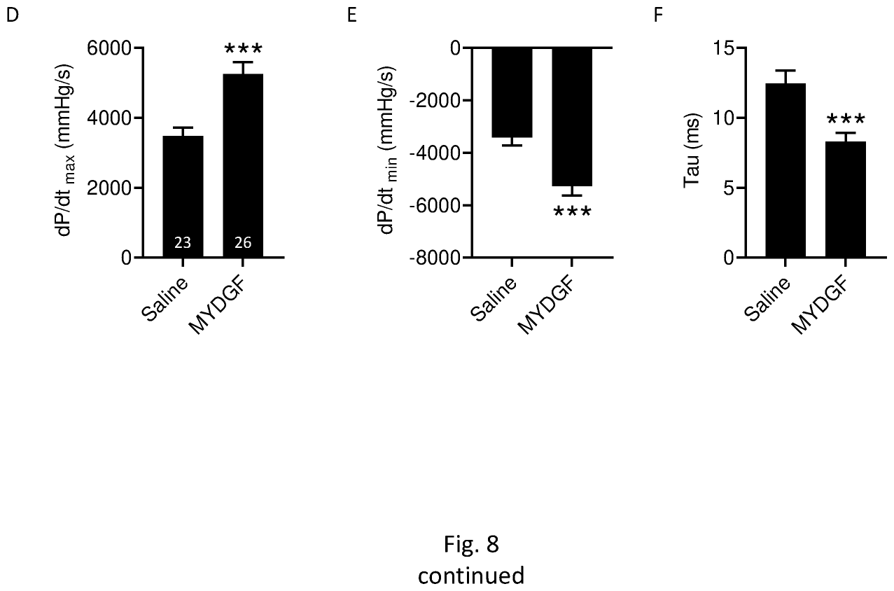

The Experiments further show that administration of MYDGF improves left ventricular systolic and diastolic functions (PV loop recordings in FIGS. 7A to 7F, and 8A to 8F).

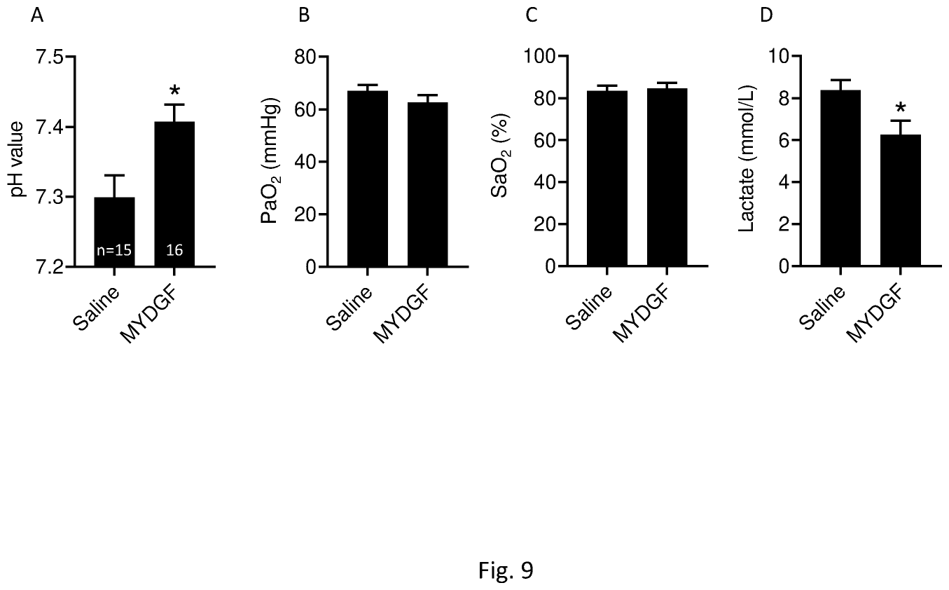

MYDGF further reverses acidosis and lowers lactate concentrations, i.e. two defining features of cardiogenic shock (blood gas analyses in FIG. 9).

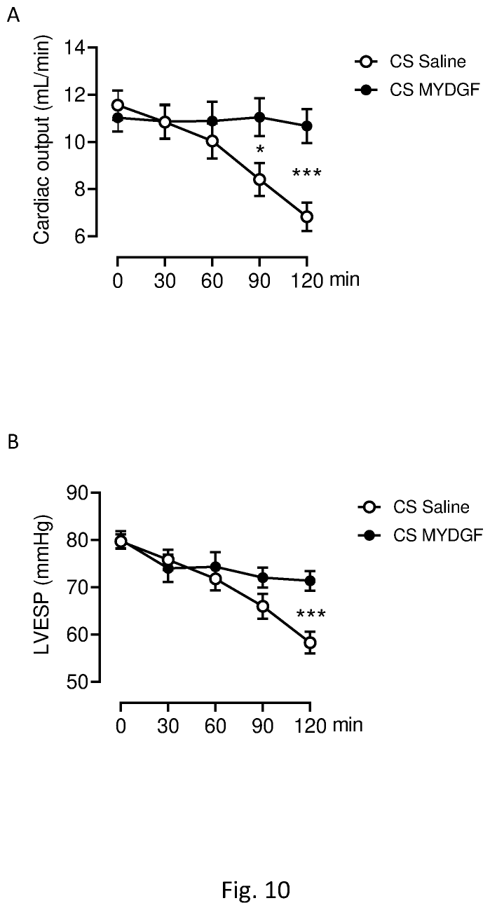

MYDGF is also shown to prevent declines in cardiac output and LVESP, i.e. two further defining features of cardiogenic shock (continuous PV loop recordings in FIGS. 10A and B).

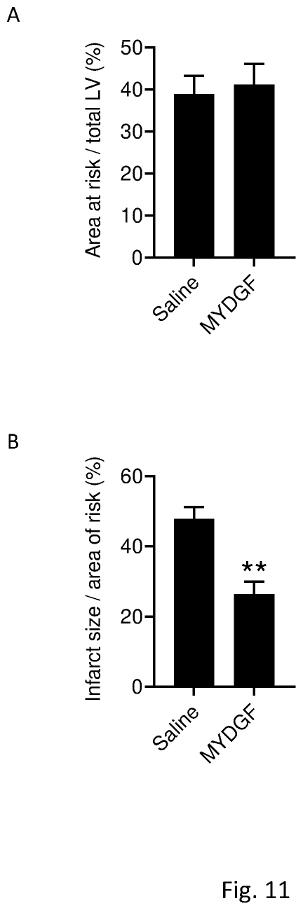

The experiments also show that treatment with MYDGF reduces infarct size (FIGS. 11A and B). For this purpose, at the end of the observation period, the left ventricle was removed and the area at risk and infarct size were measured by Evans blue and 2,3,5-triphenyltetrazolium chloride (TTC) staining (methods as described in Korf-Klingebiel et al. Nat Med. 2015; 21:140-149).

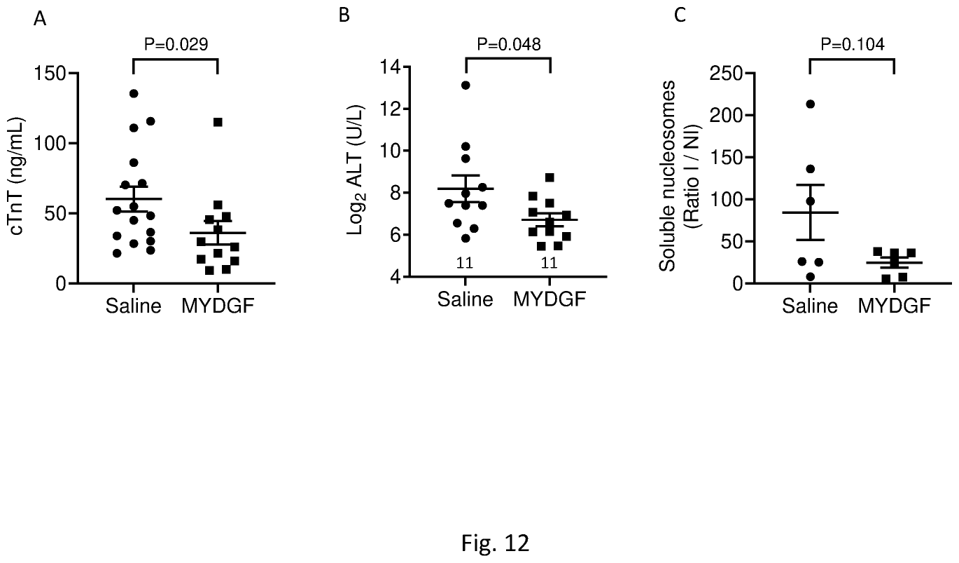

The impact of MYDGF treatment on plasma troponin levels and alanine transaminase (ALT) concentration determined at the end of the 120 minutes observation period is shown in FIGS. 12A and B. Plasma troponin is an indicator of cardiac damage and ALT reflects end-organ (liver) damage. MYDGF treatment of CS mice reduces both, plasma troponin and alanine transaminase levels. The impact of MYDGF on cell death in the infarct region was determined at the end of the 120 minutes observation period by measuring soluble nucleosomes that are released as a result of cell death. Measurements were performed using the commercially available Cell Death Detection ELISA from Roche. FIG. 12C displays the ratio of soluble nucleosomes in the infarcted region (I) over the non-infarcted region (NI) as an indicator of cell death, and shows that MYDGF significantly reduced the amount of soluble nucleosomes and thus cell death in the infarct region.

### Example 4

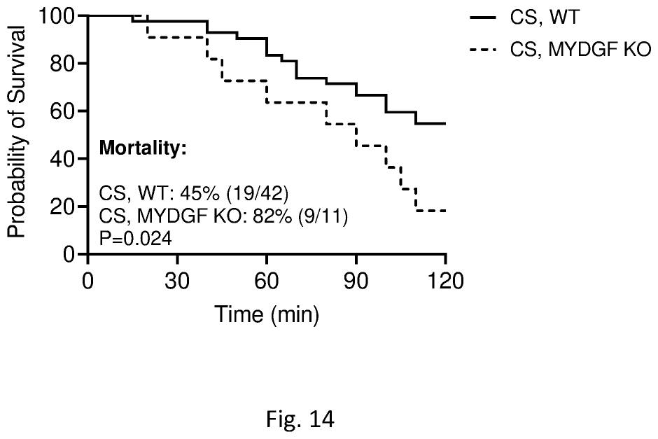

MYDGF knock-out mice were obtained as described in Korf-Klingebiel et al. (Nature Medicine, 2015, Vol. 21 (2): 140-149). Cardiogenic shock (CS) was induced in wild-type (WT) and MYDGF knock-out (KO) mice as described in Example 1. Mortality rates of WT and MYDGF knock-out mice were compared during the 120 minutes observation period. As shown in FIG. 14, mortality was significantly higher in KO mice, showing that endogenous MYDGF has a protective effect in cardiogenic shock.

### Example 5

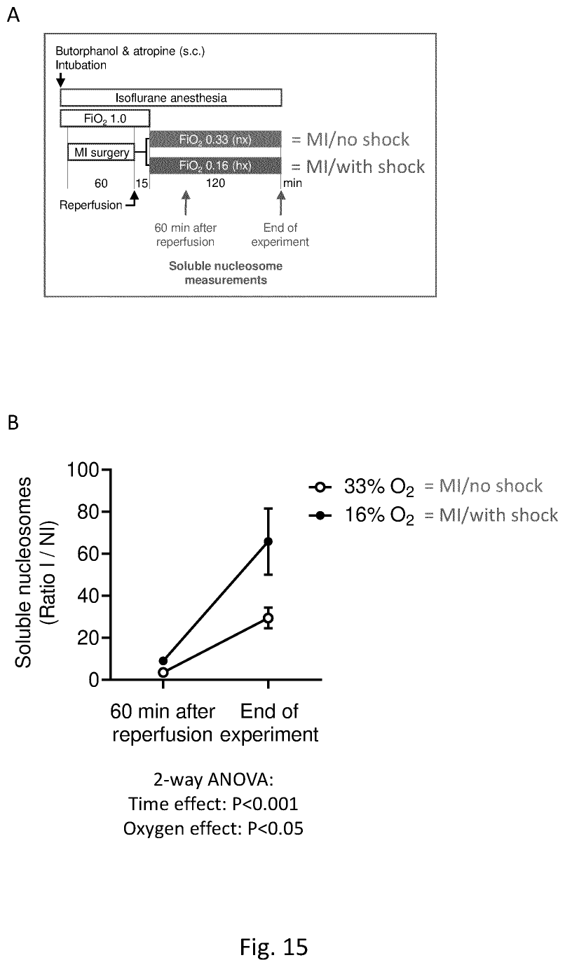

As shown e.g. in FIG. 10, heart function is continuously deteriorating during cardiogenic shock. It was assumed that this deterioration is caused by a continued aggravation of left ventricular tissue damage during cardiogenic shock. In order to test this hypothesis, acute MI was induced by transient left anterior descending coronary artery ligation for 60 min. After reperfusion, CS was induced by hypoxic ventilation (FiO₂ 0.16; MI/with shock). Infarcted control mice were normoxically ventilated (FiO₂ 0.33; MI/no shock). A scheme of the experimental set-up is shown in FIG. 15A.

60 minutes after reperfusion and at the end of the experiment, the left ventricles were removed (5 MI/no shock and 5 MI/with shock mice at each time point), and cell death (soluble nucleosomes) was measured in the infarct region (I) and the non-infarcted remote region (NI) using the cell death detection ELISA from Roche. The I/NI ratio is reported as a measure of cell death. Results are shown in FIG. 15B. Soluble nucleosome concentrations in the infarct area increased during the observation period in both groups ([apoptotic] cell death needs time to develop). The rise in soluble nucleosome concentration was significantly more pronounced in MI/with shock mice than in MI/no shock mice, indicating that CS further aggravates left ventricular tissue damage sustained during acute MI.

### Example 6

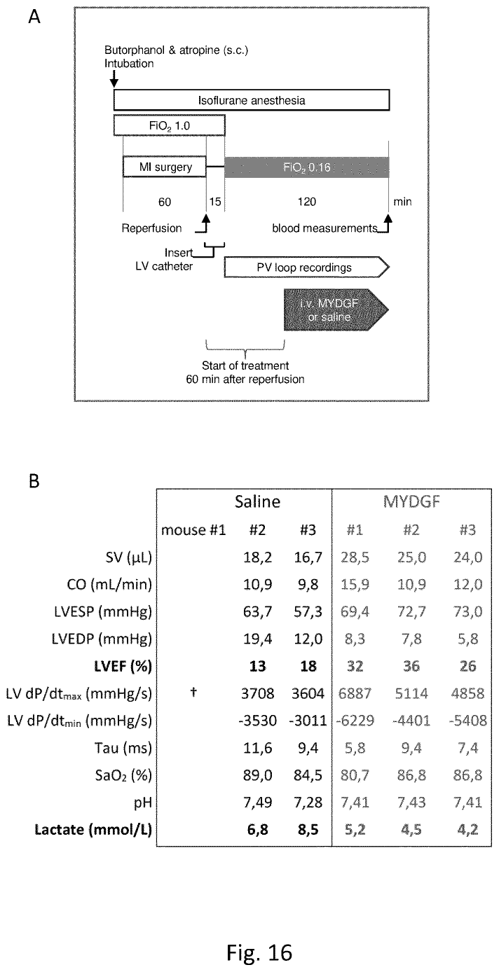

As shown in Example 5, cardiogenic shock further aggravates left ventricular tissue damage sustained during acute myocardial infarction. It was therefore tested if a delayed MYDGF therapy is capable of alleviating the symptoms of cardiogenic shock and of improving heart function. To this end, acute myocardial infarction was induced in mice by transient left anterior descending coronary artery ligation for 60 min. After reperfusion, a micromanometer-tipped conductance catheter was inserted via the right carotid artery to continuously record left ventricular pressure-volume (PV) loops. Thereafter, cardiogenic shock was induced by hypoxic ventilation (FiO₂ 0.16). Starting 60 min after reperfusion, mice received a continuous intra-left jugular vein infusion of either (i) human MYDGF (5 μg/h, infusion rate: 2 μL/min) or (ii) saline (control), until the end of the experiment when blood (anticoagulated with heparin) was drawn from the left ventricle and used for immediate blood gas and lactate analysis. Three MI/with shock mice received delayed MYDGF therapy, and three MI/with shock mice received a delayed saline infusion. A scheme of the experimental set-up is shown in FIG. 16A.

Results are shown in FIG. 16B. One of the saline-treated control mice died. In the surviving animals, heart function (e.g. left ventricular ejection fraction, LVEF) and peripheral tissue perfusion (e.g. lactate concentration) were better in MYDGF-treated mice than in saline-treated control mice. These data indicate that delayed MYDGF therapy still promotes beneficial effects in cardiogenic shock.

### Items

Items of the invention pertain to the following:

Item 1. Myeloid-derived growth factor (MYDGF) or a fragment or a variant thereof exhibiting the biological function of MYDGF, for use in treating and/or preventing cardiogenic shock.

Item 2. The MYDGF for use according to item 1, wherein the MYDGF comprises:
(i) SEQ ID NO: 1; or
(ii) a fragment or variant of SEQ ID NO: 1 exhibiting the biological function of MYDGF, wherein the variant comprises an amino acid sequence with at least 85% amino acid sequence identity to SEQ ID NO:1.

Item 3. The MYDGF for use according to item 1, wherein the MYDGF protein consists of SEQ ID NO: 1 or SEQ ID NO: 3.

Item 4. A nucleic acid encoding MYDGF or a fragment or a variant thereof exhibiting the biological function of MYDGF, for use in treating cardiogenic shock.

Item 5. The nucleic acid for use according to item 4, wherein the nucleic acid encodes an amino acid sequence having at least 85% sequence identity to SEQ ID NO:1.

Item 6. A vector comprising the nucleic acid of item 5, for use in treating and/or preventing cardiogenic shock.

Item 7. A host cell comprising the nucleic acid of item 5 or the vector according to item 6 and preferably expressing the nucleic acid, for use in treating and/or preventing cardiogenic shock.

Item 8. A pharmaceutical composition comprising the MYDGF protein or a fragment or a variant thereof exhibiting the biological function of MYDGF according to any one of items 1 to 3, the nucleic acid according to any one of items 4 or 5, the vector according to item 6, or the host cell according to item 7 for use in treating and/or preventing cardiogenic shock.

Item 9. The pharmaceutical composition for use according to item 8, wherein said pharmaceutical composition is administered through the oral, intravenous, subcutaneous, intramucosal, intraarterial, intramuscular or intracoronary route.

Item 10. The pharmaceutical composition for use of item 9, wherein the administration is through one or more bolus injection(s) and/or infusion(s).

Item 11. A method of treating and/or preventing cardiogenic shock, comprising administering to a patient in need thereof a therapeutically effective amount of MYDGF or a fragment or variant thereof exhibiting the biological function of MYDGF.

Item 12. A method of treating and/or preventing cardiogenic shock comprising administering to a patient in need thereof a therapeutically effective amount of a pharmaceutical composition comprising myeloid-derived growth factor (MYDGF) protein.

Item 13. The method according to item 11 or 12, wherein the MYDGF comprises:
(i) SEQ ID NO: 1; or
(ii) a fragment or variant of SEQ ID NO: 1 exhibiting the biological function of MYDGF, wherein the variant comprises an amino acid sequence with at least 85% amino acid sequence identity to SEQ ID NO:1.

Item 14. The method according to any one of items 11 to 13, wherein the MYDGF or fragment or variant thereof is administered through one or more bolus injection(s) and/or infusion(s), preferably in a pharmaceutically accepted carrier and/or excipient.

Item 15. A method for producing a non-human mammalian model of cardiogenic shock, the method comprising:
(i) transiently ligating a coronary artery of the mammal,
(ii) establishing reperfusion, and
(iii) ventilating the mammal with a fraction of inhaled oxygen of about 0.18 or less.

Item 16. The method of item 15, wherein the non-human mammal is a rodent, preferably wherein the non-human mammal is a mouse.

Item 17. The method of item 15 or 16, wherein the coronary artery is ligated for about 30 minutes to about 90 minutes before reperfusion is established.

Item 18. The method according to any one of items 15 to 17, wherein the mammal is ventilated with a fraction of inhaled oxygen of about 0.16.

1. Myeloid-derived growth factor (MYDGF) protein comprising SEQ ID NO: 1 or SEQ ID NO: 3, or a fragment or a variant thereof, wherein the protein or the fragment or the variant thereof exhibits the biological function of MYDGF, wherein the variant comprises an amino acid sequence with at least 85% amino acid sequence identity to SEQ ID NO: 1 or SEQ ID NO: 3, for use in treating and/or preventing cardiogenic shock.

2. The MYDGF for use according to claim 1, wherein the MYDGF comprises:
(i) SEQ ID NO: 1; or
(ii) a fragment or variant of SEQ ID NO: 1 exhibiting the biological function of MYDGF, wherein the variant comprises an amino acid sequence with at least 85% amino acid sequence identity to SEQ ID NO:1.

3. The MYDGF for use according to claim 1, wherein the MYDGF protein consists of SEQ ID NO: 1, of SEQ ID NO: 3, or of a variant of SEQ ID NO: 3 having 99% sequence identity to SEQ ID NO: 3.

4. A nucleic acid encoding MYDGF protein comprising SEQ ID NO: 1 or SEQ ID NO: 3, or a fragment or a variant thereof, wherein the protein or the fragment or the variant thereof exhibits the biological function of MYDGF, wherein the variant comprises an amino acid sequence with at least 85% amino acid sequence identity to SEQ ID NO: 1 or SEQ ID NO: 3, for use in treating and/or preventing cardiogenic shock.

5. The nucleic acid for use according to claim 4, wherein the nucleic acid encodes an amino acid sequence having at least 85% sequence identity to SEQ ID NO:1.

6. A vector comprising the nucleic acid of claim 4, for use in treating and/or preventing cardiogenic shock.

7. A host cell comprising the vector according to claim 6 and expressing the nucleic acid, for use in treating and/or preventing cardiogenic shock.

8. A pharmaceutical composition comprising the MYDGF protein or a fragment or a variant thereof according to claim 1.

9. (canceled)

10. (canceled)

11. A method of treating and/or preventing cardiogenic shock, comprising administering to a patient in need thereof a therapeutically effective amount of the MYDGF protein of claim 1.

12. A method of treating and/or preventing cardiogenic shock comprising administering to a patient in need thereof a therapeutically effective amount of the pharmaceutical composition of claim 8.

13. The method according to claim 11, wherein the MYDGF comprises:
(i) SEQ ID NO: 1; or
(ii) wherein the fragment or variant is a fragment or variant of SEQ ID NO: 1 and exhibits the biological function of MYDGF, wherein the variant comprises an amino acid sequence with at least 85% amino acid sequence identity to SEQ ID NO:1.

14. The method according to claim 11, wherein the MYDGF or fragment or variant thereof is administered through one or more bolus injection(s) and/or infusion(s), preferably in a pharmaceutically accepted carrier and/or excipient.

15. A method for producing a non-human mammalian model of cardiogenic shock, the method comprising:
(i) transiently ligating a coronary artery of the mammal,
(ii) establishing reperfusion, and
(iii) ventilating the mammal with a fraction of inhaled oxygen of about 0.18 or less.

16. The method of claim 15, wherein the non-human mammal is a rodent, preferably wherein the non-human mammal is a mouse.

17. The method of claim 15, wherein the coronary artery is ligated for about 30 minutes to about 90 minutes before reperfusion is established.

18. The method according to claim 15, wherein the mammal is ventilated with a fraction of inhaled oxygen of about 0.16.

19. A vector comprising the nucleic acid of claim 5, for use in treating and/or preventing cardiogenic shock.

20. The method of claim 12, wherein the pharmaceutical composition is administered through the oral, intravenous, subcutaneous, intramucosal, intraarterial, intramuscular or intracoronary route

21. The method according to claim 12, wherein the MYDGF comprises:
(i) SEQ ID NO: 1; or
(ii) wherein the fragment or variant is a fragment or variant of SEQ ID NO: 1 and exhibits the biological function of MYDGF, wherein the variant comprises an amino acid sequence with at least 85% amino acid sequence identity to SEQ ID NO:1.
